(19) United States
(12) Patent Application Publication
(10) Pub. No.: US 2004/0029283 A1
(43) Pub. Date: Feb. 12, 2004

(54) Intron double stranded RNA constructs and uses thereof

(76) Inventor: JoAnne J. Fillatti, Davis, CA (US)

Correspondence Address: ARNOLD & PORTER, IP DOCKETING DEPARTMENT; RM 1126(b), 555 12TH STREET, N.W., WASHINGTON, DC, 20004-1206, US

(21) Appl. No.: 10/465,800

(22) Filed: Jun. 20, 2003

Related U.S. Application Data
(60) Provisional application No. 60/390,186, filed on Jun. 21, 2002.

(51) Int. Cl. C12N 15/82; C07H 21/04

(52) U.S. Cl. 435/468; 536/23.6

(57) ABSTRACT

The present invention is in the field of plant genetics and provides agents capable of gene-specific silencing. The present invention specifically provides double-stranded RNA (dsRNA) agents, methods for utilizing such agents and plants containing such agents.

### CROSS-REFERENCE TO RELATED APPLICATION

This application claims the benefit under 35 U.S.C. § 119(e) of U.S. Provisional Application No. 60/390,186, filed Jun. 21, 2002, which application is herein incorporated by reference in its entirety.

### INCORPORATION OF SEQUENCE LISTING

A paper copy of the Sequence Listing and a computer readable form of the sequence listing on diskette, containing the file named “RNAi 16517266 US as filed.txt”, which is 60,564 bytes in size (measured in MS-DOS), and which was created on Jun. 19, 2003, are herein incorporated by reference.

### FIELD OF THE INVENTION

The present invention is in the field of plant genetics and provides agents capable of gene-specific silencing. The present invention specifically provides double stranded RNA (dsRNA) agents, methods for utilizing such agents and plants containing such agents.

### BACKGROUND OF THE INVENTION

Silencing of genes in plants occurs at both the transcriptional level and post-transcriptional level. Certain of these mechanisms are associated with nucleic acid homology at the DNA or RNA level (Matzke et al., Current Opinion in Genetics and Development, 11:221-227 (2001)). Double-stranded RNA molecules can induce sequence-specific silencing, referred to as RNA interference or RNAi. Fire et al., Nature, 391:806-811 (1988).

### SUMMARY OF THE INVENTION

The present invention includes and provides a nucleic acid construct comprising DNA which is transcribed into RNA that forms at least one double-stranded RNA molecule, such that one strand of the double-stranded molecule is coded by a portion of the DNA which is at least 90% identical to at least one transcribed intron of a gene.

The present invention also includes and provides a transformed cell or organism having in its genome an introduced nucleic acid construct comprising DNA which is transcribed into RNA that forms at least one double-stranded RNA molecule, such that one strand of the double-stranded molecule is coded by a portion of the DNA which is at least 90% identical to at least one transcribed intron of a gene.

The present invention further includes and provides a transformed plant having in its genome an introduced nucleic acid construct comprising DNA which is transcribed into RNA that forms at least one double-stranded RNA molecule, such that one strand of the double-stranded molecule is coded by a portion of the DNA which is at least 90% identical to at least one transcribed intron of a gene.

The present invention includes and provides a method of reducing expression of a protein encoded by a target gene in a mammal comprising introducing into a cell or organism a nucleic acid construct comprising DNA which is transcribed into RNA that forms at least one double-stranded RNA molecule, such that one strand of the double-stranded molecule is coded by a portion of the DNA which is at least 90% identical to at least one transcribed intron of a gene.

The present invention includes and provides a method of reducing expression of a protein encoded by a target gene in a plant comprising introducing into a plant genome a nucleic acid construct comprising DNA which is transcribed into RNA that forms at least one double-stranded RNA molecule, such that one strand of the double-stranded molecule is coded by a portion of the DNA which is at least 90% identical to at least one transcribed intron of a gene.

The present invention includes and provides a method of altering the expression of a target gene by inserting into a cell or organism a DNA construct for producing a double stranded RNA molecule coding for an intron within the target gene. More particularly, the nucleic acid construct comprises DNA which is transcribed into RNA that forms at least one double-stranded RNA molecule, one strand of which is coded by a portion of DNA which is at least 90% identical to at least one transcribed intron of a gene. In a preferred aspect of the invention, one strand of the double-stranded RNA molecule is at least 98%, even more preferably 100% identical, to an intron of a gene.

In one aspect of the invention, a construct for producing double-stranded RNA comprises one strand of an intron, a spliceable intron, and the complement of the intron, such that the spliceable intron provides a hairpin loop when the intron and the complement of the intron hybridize to each other.

In yet another aspect of this invention the constructs are based on introns within a FAD2 gene or a FAD3 gene.

In yet another aspect of this invention the construct comprises DNA which is transcribed into double-stranded RNA for at least two transcribed introns, e.g. introns for two or three or more genes.

Another aspect of this invention provides a transformed cell or organism having in its genome a nucleic acid construct which produces a double-stranded RNA of a gene to be suppressed, e.g., in a plant or an animal, preferably a plant, a mammal, an insect or a nematode. The present invention provides a transformed plant having in its genome a nucleic acid construct comprising DNA which is transcribed into RNA that forms at least one double-stranded RNA molecule such that one strand of the double-stranded molecule is coded by a portion of the DNA which is at least 90% identical to at least one transcribed intron of a native plant gene or a plant pest gene.

This invention also provides a method of reducing expression of a protein encoded by a target gene in a mammal comprising introducing into a mammalian cell or organism a nucleic acid construct comprising DNA which produces double-stranded RNA based on an intron within a gene to be suppressed. Another aspect of this invention provides a method of reducing expression of a protein encoded by a target gene in a plant comprising introducing into a plant cell or organism a nucleic acid construct comprising DNA which produces double-stranded RNA based on an intron within a gene to be suppressed.

### BRIEF DESCRIPTION OF THE DRAWINGS

FIG. 1 is a schematic of construct pCGN3892.

FIG. 2 is a schematic of construct pMON70674.

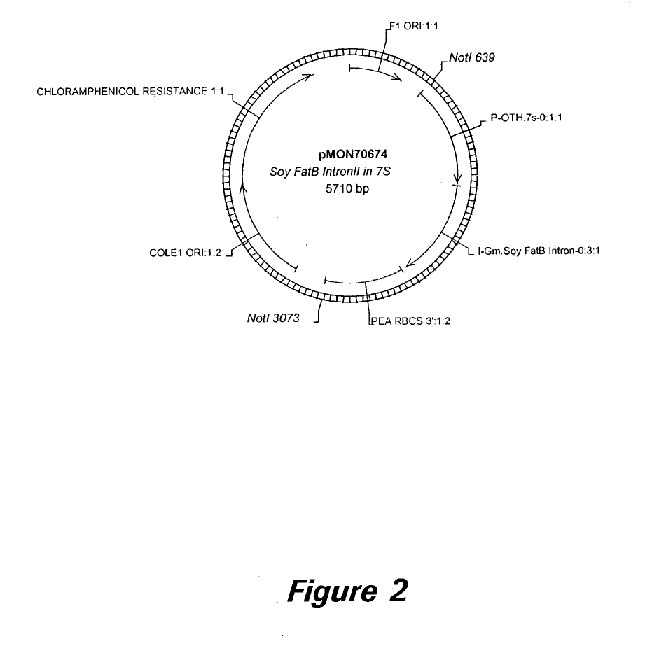

FIG. 3 is a schematic of construct pMON70678.

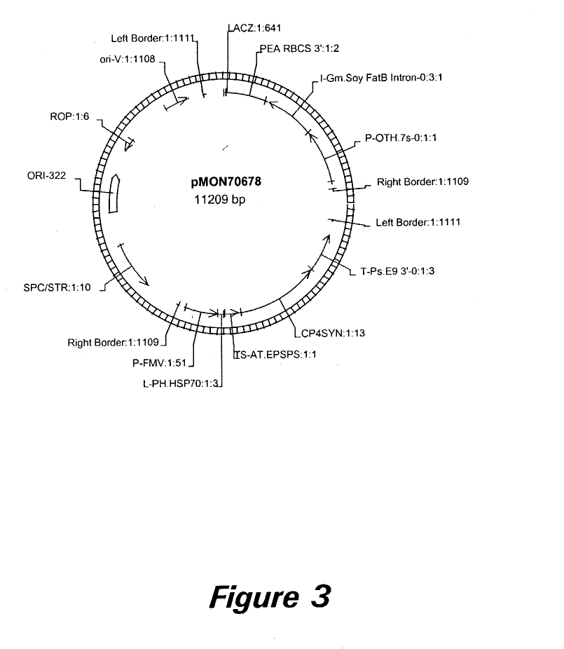

FIG. 4 is a schematic of construct pMON68546.

### DETAILED DESCRIPTION OF THE INVENTION

Description of the Nucleic Acid Sequences

SEQ ID NO: 1 sets forth a nucleic acid sequence of a FAD2-1A intron 1.

SEQ ID NO: 2 sets forth a nucleic acid sequence of a FAD2-1B intron 1.

SEQ ID NO: 3 sets forth a nucleic acid sequence of a partial FAD2-2 genomic clone.

SEQ ID NO: 4 sets forth a nucleic acid sequence of a FAD2-2B intron 1.

SEQ ID NO: 5 sets forth a nucleic acid sequence of a FAD3-1A intron 1.

SEQ ID NO: 6 sets forth a nucleic acid sequence of a FAD3-1A intron 2.

SEQ ID NO: 7 sets forth a nucleic acid sequence of a FAD3-1A intron 3A.

SEQ ID NO: 8 sets forth a nucleic acid sequence of a FAD3-1A intron 4.

SEQ ID NO: 9 sets forth a nucleic acid sequence of a FAD3-1A intron 5.

SEQ ID NO: 10 sets forth a nucleic acid sequence of a FAD3-1A intron 3B.

SEQ ID NO: 11 sets forth a nucleic acid sequence of a FAD3-1A intron 3C.

SEQ ID NO: 12 sets forth a nucleic acid sequence of a FAD3-1B intron 3C.

SEQ ID NO: 13 sets forth a nucleic acid sequence of a FAD3-1B intron 4.

SEQ ID NO: 14 sets forth a nucleic acid sequence of a FAD3-1C intron 4.

SEQ ID NO: 15 sets forth a nucleic acid sequence of a FAD2-1A gene sequence.

SEQ ID NOs: 16 and 17 set forth nucleic acid sequences of FAD2-1A PCR primers.

SEQ ID NO: 18 sets forth a nucleic acid sequence of a partial FAD2-1A genomic clone.

SEQ ID NO: 19 sets forth a nucleic acid sequence of a partial FAD2-1B genomic clone.

SEQ ID NOs: 20 and 21 set forth nucleic acid sequences of FAD3-1A PCR primers.

SEQ ID NO: 22 sets forth a nucleic acid sequence of a FAD2-1B promoter.

SEQ ID NO: 23 sets forth a nucleic acid sequence of a partial FAD3-1A genomic clone.

SEQ ID NOs: 24 through 39 set forth nucleic acid sequences of PCR primers.

SEQ ID NO: 40 sets forth a nucleic acid sequence of a soybean FATB genomic clone.

SEQ ID NO: 41 sets forth a nucleic acid sequence of a soybean FATB intron I.

SEQ ID NO: 42 sets forth a nucleic acid sequence of a soybean FATB intron II.

SEQ ID NO: 43 sets forth a nucleic acid sequence of a soybean FATB intron III.

SEQ ID NO: 44 sets forth an amino acid sequence of a soybean FATB enzyme.

SEQ ID NO: 45 sets forth a nucleic acid sequence of a soybean FATB partial genomic clone.

SEQ ID NOs: 46-53 set forth nucleic acid sequences of oligonucleotide primers.

SEQ ID NO: 54 sets forth a nucleic acid sequence of a PCR product containing soybean FATB intron II.

SEQ ID NO: 55 sets forth a nucleic acid sequence of a soybean FATB cDNA.

Definitions

As used herein, the term “gene” is used to refer to a nucleic acid sequence that encompasses a 5′ promoter region associated with the expression of the gene product, any intron and exon regions and 3′ untranslated regions associated with the expression of the gene product.

As used herein, a target gene can be any gene of interest present in an organism which contains a transcribed intron. A target gene may be endogenous or introduced.

As used herein, when referring to proteins and nucleic acids herein, the use of plain capitals, e.g., “FATB”, indicates a reference to an enzyme, protein, polypeptide, or peptide, and the use of italicized capitals, e.g., “FA TB”, is used to refer to nucleic acids, including without limitation genes, cDNAs, and mRNAs.

As used herein, a cell or organism can have a family of more than one gene encoding a particular enzyme. As used herein, a gene family is two or more genes in an organism which encode proteins that exhibit similar functional attributes. An example of two members of a gene family are FAD2-1 and FAD2-2. As used herein, a “FAD2 gene family member” is any FAD2 gene found within the genetic material of the plant. As used herein, a “FAD3 gene family member” is any FAD3 gene found within the genetic material of the plant. As used herein, a “FATB gene family member” is any FATB found within the genetic material of the plant. A gene family can be additionally classified by the similarity of the nucleic acid sequences. In a preferred aspect of this embodiment, a gene family member exhibits at least 60%, more preferably at least 70%, more preferably at least 80% nucleic acid sequence identity in the coding sequence portion of the gene.

As used herein, RNAi and dsRNA both refer to gene-specific silencing that is induced by the introduction of a double-stranded RNA molecule, see e.g., U.S. Pat. Nos. 6,506,559 and 6,573,099, and U.S. patent applications 09/056,767 and 09/127,735, all of which are incorporated herein by reference.

As used herein, a “dsRNA molecule” and an “RNAi molecule” both refer to a double-stranded RNA molecule capable, when introduced into a cell or organism, of at least partially reducing the level of an mRNA species present in a cell or a cell of an organism.

As used herein, an “intron dsRNA molecule” and an “intron RNAi molecule” both refer to a double-stranded RNA molecule capable, when introduced into a cell or organism, of at least partially reducing the level of an mRNA species present in a cell or a cell of an organism where the double-stranded RNA molecule exhibits sufficient identity to an intron of a gene present in the cell or organism to reduce the level of an mRNA containing that intron sequence.

As used herein, a “FAD2”, “A 12 desaturase” or “omega-6 desaturase” gene is a gene that encodes an enzyme capable of catalyzing the insertion of a double bond into a fatty acyl moiety at the twelfth position counted from the carboxyl terminus.

As used herein, the terminology “FAD2-1” is used to refer to a FAD2 gene that is naturally expressed in a specific manner in seed tissue.

As used herein, the terminology “FAD2-2” is used to refer a FAD2 gene that is (a) a different gene from a FAD2-1 gene and (b) is naturally expressed in multiple tissues, including the seed.

As used herein, a “FAD3”, “Δ15 desaturase” or “omega-3 desaturase” gene is a gene that encodes an enzyme capable of catalyzing the insertion of a double bond into a fatty acyl moiety at the fifteenth position counted from the carboxyl terminus.

As used herein, the terminology “FAD3-1” is used to refer a FAD3 gene that is naturally expressed in multiple tissues, including the seed.

As used herein, the capital letter that follows the gene terminology (A, B, C) is used to designate the family member, i.e., FAD2-1A is a different gene family member from FAD2-1B.

The term “non-coding” refers to sequences of nucleic acid molecules that do not encode part or all of an expressed protein. Non-coding sequences include but are not limited to introns, promoter regions, 3′ untranslated regions, and 5′ untranslated regions.

The term “intron” as used herein refers to the normal sense of the term as meaning a segment of nucleic acid molecules, usually DNA, that does not encode part of or all of an expressed protein, and which, in endogenous conditions, is transcribed into RNA molecules, but which is spliced out of the endogenous RNA before the RNA is translated into a protein. The splicing, i.e., intron removal, occurs at a defined splice site, e.g., typically at least about 4 nucleotides, between cDNA and intron sequence. For example, without limitation, the sense and antisense intron segments illustrated herein, which form a double-stranded RNA contained no splice sites.

The term “spliceable intron” as used herein refers to an intron that contains functional splice sites at each end. For example, without limitation, in the constructs illustrated herein, spliceable introns have been used to form the hairpin loop connecting two antiparallel RNA strands of intron sequence which had splice sites removed.

The term “exon” as used herein refers to the normal sense of the term as meaning a segment of nucleic acid molecules, usually DNA, that encodes part of or all of an expressed protein.

As used herein, a promoter that is “operably linked” to one or more nucleic acid sequences is capable of driving expression of one or more nucleic acid sequences, including multiple coding or non-coding nucleic acid sequences arranged in a polycistronic configuration.

As used herein, a “series” is a sequential collection of elements arranged consecutively.

Nucleic Acid Molecules

Agents of the invention include nucleic acid molecules. In an aspect of the present invention, a nucleic acid molecule comprises a nucleic acid sequence, which when introduced into a cell or organism, is capable of selectively reducing the level of a target protein and/or transcript that encodes a target protein.

In a preferred aspect, a nucleic acid molecule of the present invention exhibits sufficient homology to one or more introns which when introduced into a cell or organism as a dsRNA construct, is capable of effectively eliminating, substantially reducing, or at least partially reducing the level of an mRNA transcript or protein encoded by the gene from which the intron was derived. In another preferred aspect, a nucleic acid molecule of the present invention exhibits sufficient homology to one or more introns such that, when introduced into a cell or organism as a dsRNA construct, the nucleic acid molecule is capable of effectively eliminating, substantially reducing, or at least partially reducing the level of an mRNA transcript or protein encoded by a gene family member from which the intron was derived. In a preferred aspect, a dsRNA construct does not contain exon sequences corresponding to a sufficient part of an exon to be capable of effectively eliminating, substantially reducing, or at least partially reducing the level of an mRNA transcript or protein encoded by a gene from which the exon was derived.

An intron can be any intron from a gene, whether endogenous or introduced. Nucleic acid sequences of such introns can be derived from a multitude of sources, including, without limitation, databases such as EMBL and Genbank found at www-ebi.ac.uk/swisprot/; www-expasy.ch/; www-embl-heidelberg.de/; and www-ncbi.nlm.nih.gov. Nucleic acid sequences of such introns can also be derived, without limitation, from sources such as the GENSCAN program found at //genes.mit.edu/GENSCAN.html. In a further embodiment, additional introns may be obtained by any method by which additional introns may be identified. In a preferred embodiment, additional introns may be obtained by screening a genomic library with a probe of either known exon or intron sequences. In another preferred embodiment, additional introns may be obtained by a comparison between genomic sequence and corresponding cDNA sequence that allows identification of additional introns. In a more preferred embodiment, additional introns may be obtained by screening a genomic library with a probe of either known exon or intron sequences. The gene may then be cloned and confirmed and any additional introns may be identified by a comparison between genomic sequence and cDNA sequence. Additional introns may, for example without limitation, be amplified by PCR and used in an embodiment of the present invention.

In another preferred embodiment, an intron, such as for example, a soybean intron, may be cloned by alignment to an intron from another organism, such as, for example, Arabidopsis. In this embodiment, the location of an intron in an Arabidopsis amino acid sequence, for example, is identified. An amino acid sequence, from Arabidopsis for example, may then be aligned, with, for example a soybean amino acid sequence, providing a prediction for the location of additional soybean introns.

In a preferred aspect, the target protein is selected from the group consisting of FAD2, FAD3, and FATB. In another preferred aspect, the target protein is selected from the group of genes consisting of FAD2-1A, FAD2-1B, FAD2-2B, FAD3-1A, FAD3-1B, FAD3-1C, and FATB, or in another aspect two or more of said genes. In a preferred aspect, where homology is present between or among gene family members, at least two target proteins from the same gene family are affected. In a particularly preferred aspect, the target protein is both FAD2-1A and FAD2-1B. In another particularly preferred aspect, the target protein is both FAD3-1A and FAD3-1C.

Representative sequences for FAD2-1A, FAD2-1B, FAD2-2B, FAD3-1A, FAD3-1B, FAD3-1C introns include, without limitation, those set forth in U.S. application Ser. No. 10/176,149, filed on Jun. 21, 2002; and U.S. patent application Ser. No. 09/638,508, filed Aug. 11, 2000; and U.S. Provisional Application Serial No. 60/151,224, filed Aug. 26, 1999; and U.S. Provisional Application Serial No. 60/172,128, filed Dec. 17, 1999, all of which applications are herein incorporated by reference in their entireties including, without limitation, their accompanying sequence listings.

Representative sequences for FATB introns include, without limitation, those set forth in the present application at SEQ ID NOs: 41, 42, and 43, as well as those set forth in U.S. Pat. Nos. 5,723,761, 5,955,329, 5,955,650, 6,150,512, 6,331,664, and 6,380,462; and International Patent Publication Nos. WO 01/35726, WO 01/36598, and WO 02/15675.

Representative sequences for FATB introns also include, without limitation, those set forth in U.S. Provisional Application Serial No. 60/390,185, filed Jun. 21, 2002.

In a preferred aspect, the target protein is encoded by one member of a gene family. In another preferred aspect, the target gene is a member of a gene family. A particularly preferred use of the present invention is where two or more genes within the gene family exhibit similar nucleic acid sequences within a coding region for the target protein but exhibit dissimilar nucleic acid sequences within a transcribed intron region. In this aspect, a first nucleic acid sequence is similar to a second nucleic acid sequence if a dsRNA molecule to the first nucleic acid sequence reduces the level of a protein and/or a transcript which is encoded by the second nucleic acid sequence. Likewise, in this aspect, a first nucleic acid sequence is dissimilar to a second nucleic acid sequence if a dsRNA molecule directed to the first nucleic acid sequence does not reduce the level of a second protein and/or a transcript which is encoded by the second nucleic acid sequence.

In a preferred aspect, the target gene or target protein is a non-viral gene or protein. In another preferred aspect, the target gene or target protein is an endogenous gene or protein. In a further preferred aspect, the intron is an intron located between exons. In another preferred aspect, the intron is an intron that is within a 5′ or 3′ UTR. In another preferred aspect, the target gene or protein is a non-endogenous gene or protein; for example, the target gene or protein may be found in a plant pest, such as, for example, in a plant nematode.

Further preferred embodiments of the invention are nucleic acid molecules that are at least 85% identical, preferably at least 90% identical, more preferably 95, 97, 98, 99% identical, or most preferably 100% identical over their entire length to an intron.

“Identity,” as is well understood in the art, is a relationship between two or more polypeptide sequences or two or more nucleic acid molecule sequences, as determined by comparing the sequences. In the art, “identity” also means the degree of sequence relatedness between polypeptide or nucleic acid molecule sequences, as determined by the match between strings of such sequences. “Identity” can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J. Applied Math, 48:1073 (1988). Methods to determine identity are designed to give the largest match between the sequences tested. Moreover, methods to determine identity are codified in publicly available programs.

Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12:76-80 (1994); Birren et al., Genome Analysis, 1:543-559 (1997)). The BLASTX program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well-known Smith Waterman algorithm can also be used to determine identity.

Parameters for polypeptide sequence comparison typically include the following:

Algorithm: Needleman and Wunsch, J. Mol. Biol., 48:443-453 (1970)

Comparison matrix: BLOSSUM62 from Hentikoff and Hentikoff, Proc. Natl. Acad. Sci. USA, 89:10915-10919 (1992)

Gap Penalty: 12

Gap Length Penalty: 4

A program which can be used with these parameters is publicly available as the “gap” program from Genetics Computer Group, Madison, Wis. The above parameters along with no penalty for end gap are the default parameters for peptide comparisons.

Parameters for nucleic acid molecule sequence comparison include the following:

Algorithm: Needleman and Wunsch, J. Mol. Bio., 48:443-453 (1970)

Comparison matrix: matches−+10; mismatches=0

Gap Penalty: 50

Gap Length Penalty: 3

As used herein, “% identity” is determined using the above parameters as the default parameters for nucleic acid molecule sequence comparisons and the “gap” program from GCG, version 10.2.

The invention further relates to nucleic acid molecules that hybridize to a plant intron. In particular, the invention relates to nucleic acid molecules that hybridize under stringent conditions to the above-described nucleic acid molecules. As used herein, the terms “stringent conditions” and “stringent hybridization conditions” mean that hybridization will generally occur if there is at least 95% and preferably at least 97% identity between the sequences. An example of stringent hybridization conditions is overnight incubation at 42° C. in a solution comprising 50% formamide, 5×SSC (150 mM NaCl, 15 mM trisodium citrate), 50 mM sodium phosphate (pH 7.6), 5× Denhardt's solution, 10% dextran sulfate, and 20 micrograms/milliliter denatured, sheared salmon sperm DNA, followed by washing the hybridization support in 0.1×SSC at approximately 65° C. Other hybridization and wash conditions are well known and are exemplified in Sambrook et al., Molecular Cloning: A Laboratory Manual, Second Edition, Cold Spring Harbor, N.Y. (1989), particularly Chapter 11. As used herein, two nucleic acid molecules are said to be capable of specifically hybridizing to one another if the two molecules are capable of forming an anti-parallel, double-stranded nucleic acid structure.

One subset of the nucleic acid molecules of the invention includes fragment nucleic acid molecules. For example, fragment nucleic acid molecules may consist of significant portion(s) of, or indeed most of, a plant intron. Alternatively, fragments may comprise smaller oligonucleotides having from about 15 to about 400 contiguous nucleotide residues and more preferably, about 15 to about 45 contiguous nucleotide residues, about 20 to about 45 contiguous nucleotide residues, about 15 to about 30 contiguous nucleotide residues, about 21 to about 30 contiguous nucleotide residues, about 21 to about 25 contiguous nucleotide residues, about 21 to about 24 contiguous nucleotide residues, about 19 to about 25 contiguous nucleotide residues, or about 21 contiguous nucleotides. In a preferred embodiment, a fragment shows 100% identity to the plant intron. In another preferred embodiment, a fragment comprises a portion of a larger nucleic acid sequence.

In another aspect, a fragment nucleic acid molecule has a nucleic acid sequence that is at least 15, 25, 50, or 100 contiguous nucleotides of a nucleic acid molecule of the present invention. In a preferred embodiment, a nucleic acid molecule has a nucleic acid sequence that is at least 15, 25, 50, or 100 contiguous nucleotides of a plant intron.

In one aspect of the present invention the nucleic acids of the present invention are said to be introduced nucleic acid molecules. A nucleic acid molecule is said to be “introduced” if it is inserted into a cell or organism as a result of human manipulation, no matter how indirect. Examples of introduced nucleic acid molecules include, but are not limited to, nucleic acids that have been introduced into cells via transformation, transfection, injection, and projection, and those that have been introduced into an organism via methods including, but not limited to, conjugation, endocytosis, and phagocytosis. The cell or organism can be, or can be derived from, a plant, plant cell, algae, algae cell, fungus, fungal cell, or bacterial cell. A nucleic acid molecule of the present invention may be stably integrated into a nuclear, chloroplast or mitochondrial genome, preferably into the nuclear genome.

An agent, preferably a dsRNA molecule, is preferably capable of providing at least a partial reduction, more preferably a substantial reduction, or most preferably effective elimination of another agent such as a protein or mRNA.

As used herein, “a reduction” of the level of an agent such as a protein or mRNA means that the level is reduced relative to a cell or organism lacking a dsRNA molecule capable of reducing the agent.

As used herein, “at least a partial reduction” of the level of an agent such as a protein or mRNA means that the level is reduced at least 25% relative to a cell or organism lacking a dsRNA molecule capable of reducing the agent.

As used herein, “a substantial reduction” of the level of an agent such as a protein or mRNA means that the level is reduced relative to a cell or organism lacking a dsRNA molecule capable of reducing the agent, where the reduction of the level of the agent is at least 75%.

As used herein, “an effective elimination” of an agent such as a protein or mRNA is relative to a cell or organism lacking a dsRNA molecule capable of reducing the agent, where the reduction of the level of the agent is greater than 95%.

An agent, preferably a dsRNA molecule, is preferably capable of providing at least a partial reduction, more preferably a substantial reduction, or most preferably effective elimination of another agent such as a protein or mRNA, wherein the agent leaves the level of a second agent essentially unaffected, substantially unaffected, or partially unaffected.

As used herein, “essentially unaffected” refers to a level of an agent such as a protein or mRNA transcript that is either not altered by a particular event or altered only to an extent that does not affect the physiological function of that agent. In a preferred aspect, the level of the agent that is essentially unaffected is within 20%, more preferably within 10%, and even more preferably within 5% of the level at which it is found in a cell or organism that lacks a nucleic acid molecule capable of selectively reducing another agent.

As used herein, “substantially unaffected” refers to a level of an agent such as a protein or mRNA transcript in which the level of the agent that is substantially unaffected is within 49%, more preferably within 35%, and even more preferably within 24% of the level at which it is found in a cell or organism that lacks a nucleic acid molecule capable of selectively reducing another agent.

As used herein, “partially unaffected” refers to a level of an agent such as a protein or mRNA transcript in which the level of the agent that is partially unaffected is within 80%, more preferably within 65%, and even more preferably within 50% of the level at which it is found in a cell or organism that lacks a nucleic acid molecule capable of selectively reducing another agent.

When levels of an agent are compared, such a comparison is preferably carried out between organisms with a similar genetic background. In another even more preferable aspect, a similar genetic background is a background where the organisms being compared are plants, and the plants are isogenic except for any genetic material originally introduced using plant transformation techniques.

In a preferred aspect, the capability of a nucleic acid molecule to reduce or selectively reduce the level of a gene relative to another gene is carried out by a comparison of levels of mRNA transcripts. As used herein, mRNA transcripts include processed and non-processed mRNA transcripts. In another preferred aspect, the capability of a nucleic acid molecule to reduce or selectively reduce the level of a gene relative to another gene is carried out by a comparison of phenotype. In a preferred aspect, the comparison of phenotype is a comparison of oil composition.

In a further embodiment, a nucleic acid molecule, when introduced into a cell or organism, selectively reducing the level of a protein and/or transcript encoded by a first gene while leaving the level of a protein and/or transcript encoded by a second gene partially unaffected, substantially unaffected, or essentially unaffected, also alters the oil composition of the cell or organism.

Organisms

The constructs of this invention can be used to suppress any gene containing unique intron sequence of a target gene for suppression in a eukaryotic organism, such as for example without limitation, plants or animals, such as mammals, insects, nematodes, fish, and birds. The target gene for suppression can be an endogenous gene or a transgene in an organism to be transformed with a construct of the present invention. Alternatively, the target gene for suppression can be in a non-transgenic organism which acquires the dsRNA or DNA producing dsRNA by ingestion or infection by a transgenic organism. See e.g., U.S. Pat. No. 6,506,559.

Thus, an aspect of this invention provides a method where the target gene for suppression encodes a protein in an insect or nematode which is a pest to a plant. In an aspect, a method comprises introducing into the genome of a pest-targeted plant a nucleic acid construct comprising DNA which is transcribed into RNA that forms at least one double-stranded RNA molecule which is effective for reducing expression of a target gene within the pest when the pest, e.g., insect or nematode ingests cells from said plant. In a preferred embodiment, the gene suppression is fatal to the pest.

Plant Constructs and Plant Transformants

Exogenous genetic material may be transferred into a plant cell and the plant cell regenerated into a whole, fertile or sterile plant or plant part. Exogenous genetic material is any genetic material, whether naturally occurring or otherwise, from any source that is capable of being inserted into any organism. Such exogenous genetic material includes, without limitation, nucleic acid molecules that encode a dsRNA molecule of the present invention.

In a preferred aspect, a plant cell or plant of the present invention includes a nucleic acid molecule that exhibits sufficient homology to one or more plant introns such that when it is expressed as a dsRNA construct, it is capable of effectively eliminating, substantially reducing, or at least partially reducing the level of an mRNA transcript or protein encoded by the gene from which the intron was derived or any gene which has an intron with homology to the target intron.

In one embodiment of the invention, the expression level of a protein or transcript in one family member of that gene is selectively reduced while leaving the level of a protein or transcript of a second family member partially unaffected. In a preferred embodiment of the invention, the expression level of a protein or transcript in one family member of that gene is selectively reduced while leaving the level of a protein or transcript of a second family member substantially unaffected. In a highly preferred embodiment of the invention, the expression level of a protein or transcript in one family member of that gene is selectively reduced while leaving the level of a protein or transcript of a second family member essentially unaffected.

In a particularly preferred embodiment, a transgenic plant includes a nucleic acid molecule that comprises a nucleic acid sequence, which is capable of selectively reducing the expression level of a protein and/or transcript encoded by certain FAD2 and/or FAD3 genes while leaving the level of a protein and/or transcript of at least one other FAD2 or FAD3 gene in the plant partially unaffected or more preferably substantially or essentially unaffected.

The levels of target products such as transcripts or proteins may be decreased throughout an organism such as a plant or mammal, or such decrease in target products may be localized in one or more specific organs or tissues of the organism. For example, the levels of products may be decreased in one or more of the tissues and organs of a plant including without limitation: roots, tubers, stems, leaves, stalks, fruit, berries, nuts, bark, pods, seeds and flowers. A preferred organ is a seed.

The present invention provides nucleic acid constructs that encode a dsRNA molecule of the present invention. In a preferred aspect, such constructs comprise at least one sequence that when transcribed is a sense sequence that exhibits sufficient identity to an intron which when expressed in the presence of its complement (antisense) forms a double-stranded RNA molecule capable of at least partially reducing the level of an mRNA containing the intron sequence. In another preferred aspect, such constructs comprise at least one sequence that when transcribed is a sense sequence that exhibits sufficient identity to more than one intron, preferably more than two introns, more preferably more than three introns, which when expressed in the presence of their complements (antisense) forms a double-stranded RNA molecule capable of at least partially reducing the level of all mRNAs containing the intron sequence.

In one aspect, e.g. for suppressing plant genes, the nucleic acid construct comprises a plant promoter and a DNA sequence capable of expressing a first RNA that exhibits identity to a transcribed intron of a plant gene and expressing a second RNA capable of forming a double-stranded RNA molecule with said first RNA. In a preferred aspect, the first RNA exhibits identity to at least two, more preferably at least three or at least four, five or six plant introns. In another preferred aspect, the first RNA and the second RNA are encoded by physically linked nucleic acid sequences.

When physically linked, the nucleic acid sequences which encode the first RNA and the second RNA (the complement of the first RNA) can in a preferred aspect be separated by a sequence (spacer sequence), preferably one that promotes the formation of a dsRNA molecule. Examples of such sequences include those set forth in Wesley et al., supra, and Hamilton et al., Plant J., 15:737-746 (1988) which are capable of forming a hairpin loop between hybridized RNA. In a preferred aspect, the separating sequence is a spliceable intron. Spliceable introns include, but are not limited to, an intron selected from the group consisting of Pdk intron, FAD3 intron #5, FAD3 intron #1, FAD3 intron #3A, FAD3 intron #3B, FAD3 intron #3C, FAD3 intron #4, FAD3 intron #5, FAD2 intron #1, FAD2-2 intron. Preferred spliceable introns include, but are not limited to, an intron selected from the group consisting of FAD3 intron #1, FAD3 intron #3A, FAD3 intron #3B, FAD3 intron #3C, and FAD3 intron #5. Other preferred spliceable introns include, but are not limited to, a spliceable intron that is about 0.75 kb to about 1.1 kb in length and is capable of facilitating an RNA hairpin structure. One non-limiting example of a particularly preferred spliceable intron is FAD3 intron #5.

In a particularly preferred aspect, the construct comprises a nucleic acid where a first RNA exhibits identity to two or more, preferably three or more introns where the introns are selected from the group consisting of FAD2-1A, FAD2-1B, FAD2-2B, FAD3-1A, FAD3-1B, FAD3-1C, and FATB introns.

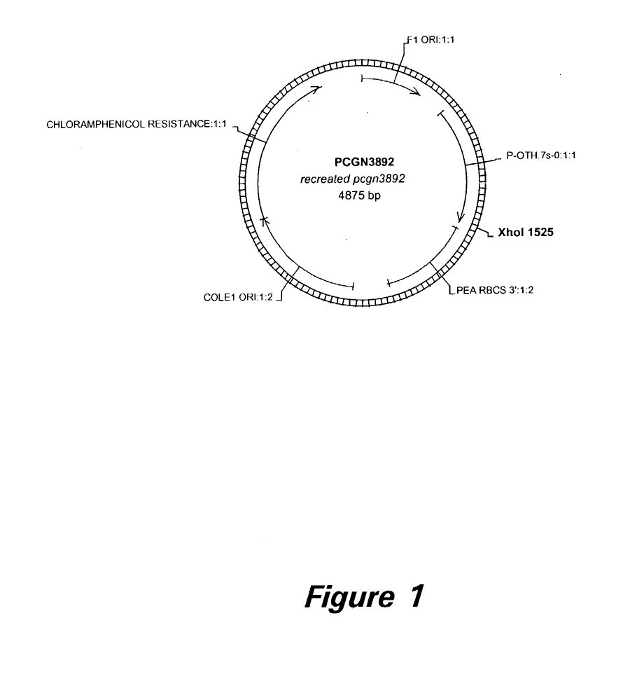

Constructs may be designed, without limitation, in a 7S expression cassette such as the pCGN3892 vector (FIG. 1). Particularly preferred constructs include the following pCGN3892 derived constructs: (1) 7S promoter—FAD2-1A sense intron—FAD3-1C sense intron—FAD3-1A sense intron FAD3-1B sense intron—spliceable FAD3 intron #5—FAD3-1B antisense intron—FAD3-1A antisense intron—FAD3-1C antisense intron—FAD2-1A antisense intron—pea rbcS; (2) 7S promoter—FAD2-1A sense intron—FAD3-1A sense intron—FAD3-1B sense intron—spliceable FAD3 intron #5—FAD3-1B antisense intron—FAD3-1A antisense intron—FAD2-1A antisense intron—pea rbcS; (3) 7S promoter—FAD2-1A sense intron—FAD3-1A sense intron—spliceable FAD3 intron #5—FAD3-1A antisense intron—FAD2-1A antisense intron—pea rbcS; (4) 7S promoter—FAD2-1A sense intron—spliceable FAD3 intron #5—FAD2-1A antisense intron—pea rbcS; (5) 7S promoter—FAD3-1A sense intron—spliceable FAD3 intron #5—FAD3-1A antisense intron—pea rbcS; (6) 7S promoter—FAD2-1A sense intron—FAD3-1A sense 3′UTR—spliceable FAD3 intron #5—FAD3-1A antisense 3′UTR—FAD2-1A antisense intron—pea rbcS; and (7) 7S promoter—FAD2-1A sense intron—FAD3-1A sense 3′UTR—FAD3-1B sense 3′UTR—spliceable FAD3 intron #5—FAD3-1B antisense 3′UTR—FAD3-1A antisense 3′UTR—FAD2-1A antisense intron—pea rbcS.

Other preferred constructs may be prepared using one or more FATB introns in a 7S expression cassette such as the pCGN3892 vector (FIG. 1). For example, other particularly preferred constructs include without limitation the following pCGN3892 derived constructs: (1) 7S promoter—FATB sense intron I—FATB sense intron II—spliceable FAD3 intron #5—FATB antisense intron II—FATB antisense intron I—pea rbcS; (2) 7S promoter—FATB sense intron II—FATB sense intron I—spliceable FAD3 intron #5—FATB antisense intron I—FATB antisense intron II—pea rbcS; (3) 7S promoter—FATB sense intron—spliceable FAD3 intron #5—FATB antisense intron—pea rbcS.

In another embodiment of the present invention, a construct lacking a promoter and a 3′ flanking region may be injected directly into either the cytoplasm, or preferably into the nucleus, of a cell via microinjection.

Transgenic DNA constructs used for transforming plant cells for intron-based RNAi will comprise the heterologous DNA which encodes the double-stranded RNA and a promoter to express the heterologous DNA in the host plant cells. As is well known in the art, such constructs typically also comprise a promoter and other regulatory elements, 3′ untranslated regions (such as polyadenylation sites), transit or signal peptides and marker genes elements as desired. For instance, see U.S. Pat. Nos. 5,858,642 and 5,322,938 which disclose versions of the constitutive promoter derived from cauliflower mosaic virus (CaMV35S), U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold-inducible promoters, U.S. Pat. No. 6,294,714 which discloses light-inducible promoters, U.S. Pat. No. 6,140,078 which discloses salt-inducible promoters, U.S. Pat. No. 6,252,138 which discloses pathogen-inducible promoters, U.S. Pat. No. 6,175,060 which discloses phosphorus deficiency-inducible promoters, U.S. Patent Application Publication 2002/0192813A1 which discloses 5′, 3′ and intron elements useful in the design of effective plant expression vectors, U.S. patent application Ser. No. 09/078,972 which discloses a coixin promoter, U.S. patent application Ser. No. 09/757,089 which discloses a maize chloroplast aldolase promoter, all of which are incorporated herein by reference.

Constructs or vectors may also include, with the region of interest, a nucleic acid sequence that acts, in whole or in part, to terminate transcription of that region. A number of such sequences have been isolated, including the Tr7 3′ sequence and the NOS 3′ sequence (Ingelbrecht et al., The Plant Cell 1:671-680 (1989); Bevan et al., Nucleic Acids Res. 11:369-385 (1983)). Regulatory transcript termination regions can be provided in plant expression constructs of this invention as well. Transcript termination regions can be provided by the DNA sequence encoding the gene of interest or a convenient transcription termination region derived from a different gene source, for example, the transcript termination region that is naturally associated with the transcript initiation region. The skilled artisan will recognize that any convenient transcript termination region that is capable of terminating transcription in a plant cell can be employed in the constructs of the present invention.

A vector or construct may also include regulatory elements. Examples of such include the Adh intron 1 (Callis et al., Genes and Develop. 1:1183-1200 (1987)), the sucrose synthase intron (Vasil et al., Plant Physiol. 91:1575-1579 (1989)) and the TMV omega element (Gallie et al., The Plant Cell 1:301-311 (1989)). These and other regulatory elements may be included when appropriate.

In practice DNA is introduced into only a small percentage of target cells in any one experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Potentially transformed cells are exposed to the selective agent. In the population of surviving cells will be those cells where, generally, the resistance-conferring gene has been integrated and expressed at sufficient levels to permit cell survival. Cells may be tested further to confirm stable integration of exogenous DNA. Useful selective marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable markers are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Transformation Methods and Transgenic Plants

Methods and compositions for transforming plants by introducing a transgenic DNA construct or a nucleic acid molecule of the present invention into a plant genome in the practice of this invention can include any of the well-known and demonstrated methods. Preferred methods of plant transformation are microprojectile bombardment as illustrated in U.S. Pat. Nos. 5,015,580; 5,550,318; 5,538,880; 6,160,208; 6,399,861 and 6,403,865 and Agrobacterium-mediated transformation as illustrated in U.S. Pat. Nos. 5,635,055; 5,824,877; 5,591,616; 5,981,840 and 6,384,301, all of which are incorporated herein by reference. See also U.S. patent application Ser. No. 09/823,676, incorporated herein by reference, for a description of vectors, transformation methods, and production of transformed Arabidopsis thaliana plants where transcription factors such as G1073 are constitutively expressed by a CaMV35S promoter.

Transformation methods of this invention to provide plants with enhanced environmental stress tolerance are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, Type I, Type II, and Type III callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells, which are capable of proliferating as calli, also are recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. No. 6,194,636 and U.S. patent application Ser. No. 09/757,089, which are incorporated herein by reference.

Examples of species that have been transformed by microprojectile bombardment include monocot species such as maize (PCT Publication WO 95/06128), barley, wheat (U.S. Pat. No. 5,563,055, specifically incorporated herein by reference in its entirety), rice, oat, rye, sugarcane, and sorghum; as well as a number of dicots including tobacco, soybean (U.S. Pat. No. 5,322,783, specifically incorporated herein by reference in its entirety), sunflower, peanut, cotton, tomato, and legumes in general (U.S. Pat. No. 5,563,055, specifically incorporated herein by reference in its entirety).

The regeneration, development, and cultivation of plants from various transformed explants is well documented in the art. This regeneration and growth process typically includes the steps of selecting transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Cells that survive the exposure to the selective agent, or cells that have been scored positive in a screening assay, may be cultured in media that supports regeneration of plants. Developing plantlets are transferred to soil less plant growth mix, and hardened off, prior to transfer to a greenhouse or growth chamber for maturation.

The present invention can be used with any transformable cell or tissue. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

Any suitable plant culture medium can be used. Examples of suitable media would include but are not limited to MS-based media (Murashige and Skoog, Physiol. Plant, 15:473-497, (1962) or N6-based media (Chu et al., Scientia Sinica 18:659, (1975) supplemented with additional plant growth regulators including but not limited to auxins, cytokinins, ABA, and gibberellins. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Those of skill in the art are aware that media and media supplements such as nutrients and growth regulators for use in transformation and regeneration and other culture conditions such as light intensity during incubation, pH, and incubation temperatures can be optimized for the particular variety of interest.

Any of the nucleic acid molecules of the invention may be introduced into a plant cell in a permanent or transient manner in combination with other genetic elements, for example, including but not limited to, vectors, promoters, and enhancers. Further, any of the nucleic acid molecules of the invention may be introduced into a plant cell in a manner that allows for expression or overexpression of the protein or fragment thereof encoded by the nucleic acid molecule.

It is understood that two or more nucleic molecules of the present invention may be introduced into a plant using a single construct and that construct can contain more than one promoter. In embodiments where the construct is designed to express two nucleic acid molecules, it is preferred that the two promoters are (i) two constitutive promoters, (ii) two seed-specific promoters, or (iii) one constitutive promoter and one seed-specific promoter. Preferred seed-specific and constitutive promoters are a napin and a 7S promoter, respectively. It is understood that two or more of the nucleic molecules may be physically linked and expressed utilizing a single promoter, preferably a seed-specific or constitutive promoter.

It is further understood that two or more nucleic acids of the present invention may be introduced into a plant using two or more different constructs. Alternatively, two or more nucleic acids of the present invention may be introduced into two different plants and the plants may be crossed to generate a single plant expressing two or more nucleic acids. In an RNAi embodiment, it is understood that the sense and antisense strands may be introduced into the same plant on one construct or two constructs. Alternatively, the sense and antisense strands may be introduced into two different plants and the plants may be crossed to generate a single plant expressing both sense and antisense strands.

The present invention also provides for parts of the plants, particularly reproductive or storage parts. Plant parts, without limitation, include seed, endosperm, ovule, pollen, roots, tubers, stems, leaves, stalks, fruit, berries, nuts, bark, pods, seeds and flowers. In a particularly preferred embodiment of the present invention, the plant part is a seed.

The present invention also provides a container of over 10,000, more preferably 20,000, and even more preferably 40,000 seeds where over 10%, more preferably 25%, more preferably 50% and even more preferably 75% or 90% of the seeds are seeds derived from a plant of the present invention.

The present invention also provides a container of over 10 kg, more preferably 25 kg, and even more preferably 50 kg seeds where over 10%, more preferably 25%, more preferably 50% and even more preferably 75% or 90% of the seeds are seeds derived from a plant of the present invention.

Plants of the present invention can be part of or generated from a breeding program. The choice of breeding method depends on the mode of plant reproduction, the heritability of the trait or traits being improved, and the type of cultivar used commercially (e.g., F₁ hybrid cultivar, pureline cultivar, etc). Selected, non-limiting approaches, for breeding the plants of the present invention are set forth below. A breeding program can be enhanced using marker-assisted selection of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of genetic worth. A breeder can select and cross two or more parental lines, followed by repeated selfing and selection, producing many new genetic combinations.

The development of new cultivars requires the development and selection of varieties, the crossing of these varieties and the selection of superior hybrid crosses. The hybrid seed can be produced by manual crosses between selected male-fertile parents or by using male sterility systems. Hybrids are selected for certain single gene traits such as pod color, flower color, seed yield, pubescence color, or herbicide resistance, which indicate that the seed is truly a hybrid. Additional data on parental lines, as well as the phenotype of the hybrid, influence a breeder's decision whether to continue with the specific hybrid cross.

Agents of the present invention can be utilized in a variety of methods. For example, the present invention provides a method of altering the expression of a target gene comprising (a) introducing into a cell a first DNA sequence capable of expressing a first RNA which exhibits identity to a transcribed intron of the target gene and a second DNA sequence and a method of modifying a level of a target protein comprising: (a) growing a plant having integrated into a genome a nucleic acid molecule comprising a first DNA sequence which encodes a first RNA that exhibits identity to a transcribed intron of an mRNA that encodes the target protein and a second DNA sequence capable of expressing a second RNA capable of forming a double-stranded RNA molecule with the first RNA and (b) expressing the first and second RNA. In a preferred aspect, the expression of a target gene is altered or modified if the level of an mRNA or protein encoded by that gene is altered, in a more preferred aspect, a method of the present invention provides for at least a partial reduction, or more preferably a substantial reduction or effective elimination of an encoded agent such as a protein or mRNA.

The following examples are illustrative and not intended to be limiting in any way.

### EXAMPLES

### Example 1

### This Example Illustrates the Identification of Introns Which are Useful for Demonstrating the Suppression of Genes Using Intron Double-Stranded RNA Molecules

1A. Soybean A12 Desalurase (FAD2-1)

A soybean FAD2-1A sequence is identified by screening a soybean genomic library using a soybean FAD2-1 cDNA probe. Three putative soy FAD2-1 clones are identified and plaque purified. Two of the three soy FAD2-1 clones are ligated into pBluescript II KS+ (Stratagene) and sequenced. Both clones (14-1 and 11-12) are the same and match the soy FAD2-1 cDNA exactly. A sequence of the entire FAD2-1A clone is provided in SEQ ID NO:15.

Prior to obtaining a full length clone, a portion of the FAD2-1A genomic clone is PCR amplified using PCR primers designed from the 5′ untranslated sequence (Primer 12506, 5′-ATACAA GCCACTAGGCAT-3′, SEQ ID NO:16) and within the cDNA (Primer 11698: 5′-GATTGGCCATGCAATGAGGGAAAAGG-3′, SEQ ID NO:17). The resulting PCR product is cloned into the vector pCR 2.1 (Invitrogen) and sequenced. A soy FAD2-1A partial genomic clone (SEQ ID NO:18) with an intron region (SEQ ID NO:1) is identified by comparison to the soybean cDNA sequence using the Pustell comparison program in Macvector. The FAD2-1A intron #1 sequence (SEQ ID NO:1) begins after the ATG start codon, and is 420 bases long.

A second FAD2-1 gene family member is also identified and cloned, and is referred to herein as FAD2-1B. The soy FAD2-1B partial genomic clone (SEQ ID NO:19) has a coding region (base pairs 1783-1785 and 2191-2463) and an intron region (base pairs 1786-2190) which are identified by comparison to the soybean cDNA sequence using the Pustell comparison program in Macvector. The FAD2-1B intron #1 sequence (SEQ ID NO:2) begins after the ATG start codon and is 405 bases long. Other regions in the FAD2-1B partial genomic clone (SEQ ID NO: 19) include a promoter (base pairs 1-1704) (SEQ ID NO: 22) and 5′UTR (base pairs 1705-1782).

1B. Soybean A15 Desaturase (FAD3)

A partial soybean FAD3-1A genomic sequence is PCR amplified from soybean DNA using primers 10632, 5′-CUACUACUACUACTCGAGACAAAGCCTTTAGCCTATG-3′ (SEQ ID NO: 20), and 10633: 5′-CAUCAUCAUCAUGGATCCCATGTCTCTCTATGCAAG-3′ (SEQ ID NO: 21). The Expand Long Template PCR system (Roche Applied Sciences, Indianapolis) is used according to the manufacturer's directions. The resulting PCR products are cloned into the vector pCR 2.1 (Invitrogen) and sequenced. A soy FAD3-1A partial genomic clone sequence (SEQ ID NO: 23) and intron regions are confirmed by comparisons to the soybean FAD3-1A cDNA sequence using the Pustell program in Macvector.

From the identified partial genomic soybean FAD3-1A sequence (SEQ ID NO:23), seven introns are identified: FAD3-1A intron #1 (SEQ ID NO:5), FAD3-1A intron #2 (SEQ ID NO:6), FAD3-1A intron #3A (SEQ ID NO:7), FAD3-1A intron #4 (SEQ ID NO:8), FAD3-1A intron #5 (SEQ ID NO:9), FAD3-1A intron #3B (SEQ ID NO:10), and FAD3-1A intron #3C (SEQ ID NO:11). FAD3-1A intron #1 is 191 base pairs long and is located between positions 294 and 484, FAD3-1A intron #2 is 346 base pairs long and is located between positions 577 and 922, FAD3-1A intron #3A is 142 base pairs long and is located between positions 991 and 1132, FAD3-1A intron #3B is 98 base pairs long and is located between positions 1224 and 1321, FAD3-1A intron #3C is 115 base pairs long and is located between positions 1509 and 1623, FAD3-1A intron #4 is 1228 base pairs long and is located between positions 1707 and 2934, and FAD3-1A intron #5 is 625 base pairs long and is located between positions 3075 and 3699.

Introns #3C and #4 are also PCR amplified from a second FAD3 gene family member (FAD3-1B). Soybean FAD3-1B introns #3C and #4 are PCR amplified from soybean DNA using the following primers, 5′CATGCTTTCTGTGCTTCTC 3′ (SEQ ID NO: 26) and 5′ GTTGATCCAACCATAGTCG 3′ (SEQ ID NO: 27). The PCR products are cloned into the vector pCR 2.1 (Invitrogen) and sequenced. Sequences for the FAD3-1B introns #3C and #4 are provided in SEQ ID NOs:12 and 13, respectively.

1C. FATB Thioesterase

A soybean FATB sequence is identified by screening a soybean genomic library using a soybean FATB cDNA probe (SEQ ID NO: 55). Leaf tissue is obtained from Asgrow soy variety A3244, ground up in liquid nitrogen and stored at −80° C. until use. 6 ml of SDS Extraction buffer (650 ml sterile ddH₂O, 100 ml 1M Tris-Cl pH 8, 100 ml 0.25M EDTA, 50 ml 20% SDS, 100 ml 5M NaCl, 4 μl beta-mercaptoethanol) is added to samples of 2 ml frozen/ground leaf tissue, and the mixture is incubated at 65° C. for 45 min. The samples are shaken every 15 min. 2 ml ice-cold 5M potassium acetate is added to each sample, the samples are shaken, and then incubated on ice for 20 min. 3 ml CHCl₃ is added to each sample, and then the samples are shaken for 10 min.

The samples are then centrifuged at 10,000 rpm for 20 min, and the protocol is continued with the supernatant. 2 ml isopropanol is added to each sample and mixed. The samples are then centrifuged at 10,000 rpm for 20 min, and the supernatant is drained. The pellet is resuspended in 200 μl RNase, and incubated at 65° C. for 20 minutes. 300 μl ammonium acetate/isopropanol (1:7) is added, and mixed. The samples are then centrifuged at 10,000 rpm for 15 minutes, and the supernatant is discarded. The pellet is rinsed with 500 l 80% ethanol, and allowed to air dry. The pellet is then resuspended in 200 μl T10E1 (10 mM Tris: 1 mM EDTA). Approximately 840 μg of clean gDNA is obtained.

Based on the FATB cDNA sequence and restriction enzyme patterns, six oligonucleotides are synthesized: F1 (SEQ ID NO: 46), F2 (SEQ ID NO: 47), F3 (SEQ ID NO: 48), R1 (SEQ ID NO: 49), R2 (SEQ ID NO: 50), and R3 (SEQ ID NO: 51). The oligonucleotide are used in pairs for PCR amplification of the isolated soy genomic DNA: pair 1 (F1+R1), pair 2 (F1+R2), pair 3 (F1+R3), pair 4 (F2+R1), pair 5 (F2+R2), pair 6 (F2+R3), pair 7 (F3+R1), and pair 8 (F3+R2). The PCR amplification is carried out as follows: 1 cycle, 95° C. for 10 min; 40 cycles, 95° C. for 1 min, 58° C. for 30 sec, 72° C. for 55 sec; 1 cycle, 72° C. for 7 min. Three positive fragments are obtained, specifically from primer pairs 3, 6, and 7. Each fragment is cloned into vector pCR2.1 (Invitrogen). Cloning is successful for fragment #3, which is confirmed and sequenced (SEQ ID NO: 45).

Three introns are identified in the soybean FATB gene by comparison of the genomic sequence to the cDNA sequence: intron I (SEQ ID NO: 41) spans base 106 to base 214 of the genomic sequence (SEQ ID NO: 45) and is 109 bp in length; intron II (SEQ ID NO: 42) spans base 289 to base 1125 of the genomic sequence (SEQ ID NO: 45) and is 837 bp in length; and intron III (SEQ ID NO: 43) spans base 1635 to base 1803 of the genomic sequence (SEQ ID NO: 45) and is 169 bp in length.

### Example 2

### This Example Illustrates Constructs for Expressing Double-Stranded RNA Using Separate Promoters for the Sense and Antisense Introns

The FAD2-1A intron #1 sequence (SEQ ID NO: 1) is amplified via PCR using the FAD2-1A partial genomic clone (SEQ ID NO: 18) as a template and primers 12701 (5′-ACGAATTCCTCGAGGTAAA TTAAATTGTGCCTGC-3′ (SEQ ID NO: 24)) and 12702 (5′-GCGAGATCTATCG ATCTGTGTCAAAGTATAAAC-3′ (SEQ ID NO: 25)). The resulting amplification products are cloned into the vector pCR 2.1 (Invitrogen) and sequenced. The FAD2-1A intron is then cloned into the expression cassette, pCGN3892 (FIG. 1), in sense and antisense orientations. The vector pCGN3892 contains the soybean 7S alpha′ promoter and a pea rbcS 3′. Both gene fusions are then separately ligated in two sequential steps into pCGN9372, a vector that contains the CP4 gene regulated by the FMV promoter. The resulting vector, which contains the FAD2-1A intron in the sense and antisense orientation driven by two separate 7S alpha′ promoters and the FMV-CP4 gene selectable marker, is transformed into soybean via Agrobacterium tumefaciens strain ABI using methods generally described by Martinell in U.S. Pat. No. 6,384,310 to provide transgenic soybean plants with the FAD2 gene suppressed.

Four of the seven introns identified from the soybean FAD3-1A genomic clone are PCR amplified using the FAD3-1A partial genomic clone as template and primers as follows: FAD3-1A intron #1, primers 12568: 5′-GATCGATGCCCGGGGTAATAATTTTTGTGT-3′ (SEQ ID NO: 30) and 12569: 5′-CACGCCTCGAGTGTTCAATTCAATCAATG-3′ (SEQ ID NO: 31); FAD3-1A intron #2, primers 12514: 5′-CACTCGAGTTAGTTCATACTGGCT-3′ (SEQ ID NO: 32) and 12515: 5′-CGCATCGATTGCAAAATCCATCAAA-3′ (SEQ ID NO: 33); FAD3-1A intron #4, primers 10926: 5′-CUACUACUACUACTCGAGCGTAAATAGTGGGTGAACAC-3′ (SEQ ID NO: 34) and 10927: 5′-CAUCAUCAUCAUCTCGAGGAATTCGTCCATTTTAGTACACC-3′ (SEQ ID NO: 35); FAD3-1A intron #5, primers 10928: 5′-CUACUACUACUACTCGAGGCGCGT ACATTTTATTGCTTA-3′ (SEQ ID NO: 36) and 10929: 5′-CAUCAUCAUCAUCT CGAGGAATTCTGCAGTGAATCCAAATG-3′ (SEQ ID NO: 37). The resulting PCR products for each intron are cloned into the vector pCR 2.1 (Invitrogen) and sequenced.

FAD3-1A introns #1, #2, #4 and #5 are all ligated separately into the pCGN3892, in sense and antisense orientations. pCGN3892 (FIG. 1) contains the soybean 7S alpha′ promoter and a pea rbcS 3′. These fusions are ligated in two sequential steps into pCGN9372, a vector that contains the CP4 gene regulated by the FMV promoter for transformation into soybean. The resulting vectors contain a sense and antisense copy of each intron driven by two separate 7S alpha′ promoters. For example, one such vector contains the FAD3-1A intron #1 in the sense and antisense orientation driven by two separate 7S alpha′ promoters and the FMV-CP4 gene selectable marker. A second example contains the FAD3-1A intron #4 in the sense and antisense orientation driven by two separate 7S alpha′ promoters and the FMV-CP4 gene selectable marker. Vectors containing such sense and antisense constructs are transformed into soybean via Agrobacterium tumefaciens strain ABI using methods generally described by Martinell in U.S. Pat. No. 6,384,310.

### Example 3

### This Example Illustrates Constructs for Expressing Double-Stranded RNA Using Separate Promoters for the Sense And Antisense Introns

The soybean FATB intron II sequence (SEQ ID NO: 42) is amplified via PCR using the FATB fragment #3 partial genomic clone (SEQ ID NO: 45) as a template and primers 18133 (SEQ ID NO: 52) and 18134 (SEQ ID NO: 53). PCR amplification is carried out as follows: 1 cycle, 95° C. for 10 min; 25 cycles, 95° C. for 30 sec, 62° C. for 30 sec, 72° C. for 30 sec; 1 cycle, 72° C. for 7 min.

PCR amplification results in a product (SEQ ID NO: 54) that is 854 bp long, including reengineered restriction sites at both ends. The FATB intron #2 PCR product is cloned separately in two sequential steps directly into the expression cassette pCGN3892 (FIG. 1) in a sense or antisense orientation. Vector pCGN3892 contains the soybean 7S alpha′promoter and a pea RBCS 3′. The resulting vector contains a sense and antisense copy of the FATB intron #2, each of which is driven by a separate 7S alpha′ promoter. The resulting gene expression construct, is used for transformation of soybean using Agrobacterium methods as described herein.

### Example 4

The following sixteen steps illustrate the construction of a vector pMON68546 designed for plant transformation to suppress FAD2, FAD3, and FA TB genes in soybean. In particular, the construct comprises a 7S alpha promoter operably linked to a series of soybean sense-oriented introns, i.e., a FAD2-1A intron #1, a FAD3-1A intron #4, a FATB intron #2, a FAD3-1B intron #4, a hairpin loop-forming spliceable intron, and a complementary series of soybean anti-sense-oriented introns, i.e., a FAD3-1B intron #4, a FATB intron #2, a FAD3-1A intron #4 and a FAD2-1A intron #1.

Step1—The soybean FAD3-1A intron #5, which serves as the spliceable intron portion of the RNAi construct, is PCR amplified using Soy genomic DNA as template, with the following primers:

5′primer=19037=ACTAGTATATTGAGCTCATATTCCACTGCAGTGGATATTGTTTAAACATAGCTAGCA TATTACGCGTATATTATACAAGCTTATATTCCCGGGATATTGTCGACATATTAGCGG TACATTTTATTGCTTATTCAC 3′ primer=19045=ACTAGTATATTGAGCTCATATTCCTGCAGGATATTCTCGAGATATTCACGGTAGTAA TCTCCAAGAACTGGTTTTGCTGCTTGTGTCTGCAGTGAATC. These primers add cloning sites to the 5′ and 3′ ends. To 5′ end: SpeI, SacI, BstXI, PmeI, NheI, MluI, HindIII, XmaI, SmaI, SalI. To 3′ end: SpeI, SacI, Sse83871, XhoI. The Soy FAD3-1A intron #5 PCR product is cloned into PCR2.1, resulting in KAWHIT03.0065.

Step 2—The soybean FAD3-1A intron #5 PCR product is then cloned into an empty AMP vector by digesting KAWHIT03.0065 (Soybean FAD3-1A intron #5 in pCR2.1) with SpeI and then the ends are filled in using the Klenow fragment of T4 Polymerase. pMON68526 (empty AMP vector) is digested with HindIII and then the ends are filled in using the Klenow fragment of T4 Polymerase. The soybean FAD3-1A PCR product with the restriction sites described above is blunt-end ligated into pMON68526, resulting in pMON68541 (FAD3-1A PCR product in empty AMP vector).

Step 3—The soybean FAD 2-1A intron #1 is PCR amplified using soybean genomic DNA as template, with the following primers:

5′ primer=18663=GGGCCCGGTAAATTAAATTGTGC (Adding Bsp120I site to 5′ end);

3′ primer=18664=CTGTGTCAAAGTATAAACAAGTTCAG. The resulting PCR product is cloned into PCR 2.1 creating KAWHIT03.0038.

Step 4—Soybean FAD 2-1A intron #1 PCR product in KAWHIT03.0038 is cloned into KAWHIT03.0032 (empty CM resistant vector with a multiple cloning site) using the restriction sites Bsp120I and EcoRI. The resulting plasmid is KAWHIT03.0039 (Soybean FAD 2-1A intron #1 in empty CM resistant vector).

Step 5—KAWHIT03.0039 is digested with AscI and HindIII and pMON68541 (FAD3-1A PCR product in empty AMP vector) is digested with MluI and HindIII. The Soybean FAD 2-1A intron #1 is then directionally cloned into pMON68541 to generate KAWHIT03.0071 (soybean FAD2-1A intron #1 with soybean FAD3-1A Intron #5).

Step 6-5′ and 3′ end portions of soybean FAD3-1A intron #4 are PCR amplified to create a 376 bp fragment using genomic DNA as template and the following primers:

5′ Primer of 5′ end=19034=GGGCCCAAATAGTGGGTGAAC (This primer added a Bsp120I site to 5′ end)

3′ Primer of 5′ end=18993=GAACTAAGGGACACAAC

5′ Primer of 3′ end=18990=CTTAGTTCGCTCTTACCTGTGATC

3′ Primer of 3′ end=18996=GTCCATTTTAGTACACCAC

The resulting PCR product is cloned into PCR 2.1 to form KAWHITO3.0067 containing the 5′ and 3′ ends of intron #4 from the soybean FAD3-1A.

Step 7—KAWHIT03.0067 is cloned into KAWHIT03.0032 (empty CM resistant vector with a multiple cloning site) using the restriction sites Bsp120I and EcoRI, resulting in plasmid KAWHIT03.0068.

Step 8—KAWHIT03.0068 (5′ and 3′ ends of intron #4 from the soybean FAD3-1A in CM resistant Vector) is digested with AscI and HindIII and KAWHIT03.0071 (Soybean FAD2-1A intron #1 with soybean FAD3-1A intron #5) is digested with MluI and HindIII. The 5′ and 3′ ends of intron #4 from the soybean FAD3-1A are directionally ligated into KAWHIT03.0071 creating KAWHIT03.0075 (soybean FAD2-1A intron#1, soybean FAD3-1A intron #4 ends and soybean FAD3-1A intron #5).

Step 9—5′ and 3′ end portions of soybean FATB intron #2 are PCR amplified to create a 374 bp fragment using genomic DNA as template and the following primers:

5′ Primer of 5′ end=19205=GGGCCCTTCTCGATTCTTTTCTC (Adding Bsp120I site to 5′ end)

3′ Primer of 5′ end=19147=CAGACAAGGCAAAGAAACAAGGGAG

5′ Primer of 3′ end=19088=GCCTTGTCTGGTCCGATTGATTTCTCG

3′ Primer of 3′ end=19089=CATGCATGCAAAATATACGCAAGTTAG The resulting PCR product is cloned into PCR 2.1 to form KAWHIT03.0069.

Step 10—KAWHIT03.0069 (containing the 5′ and 3′ ends of Intron #2 from the soybean FATB) is cloned into KAWHIT03.0032 (empty CM resistant vector with a multiple cloning site) using the restriction sites Bsp1201 and EcoRI to create KAWHIT03.0070. (5′ and 3′ ends of intron #2 from the soybean FATB in CM resistant vector).

Step 11—KAWHIT03.0070 (5′ and 3′ ends of intron #2 from the soybean FATB in CM resistant vector) is digested with AscI and HindIII and KAWHIT03.0075 (Soybean FAD2-1A intron #1, soybean FAD3-1A intron #4 ends and soybean FAD3-1A intron #5) is digested with MluI and HindIII. The 5′ and 3′ ends of intron #2 from the soybean FATB are directionally ligated into KAWHIT03.0075 to generate KAWHIT03.0077 (Soybean FAD2-1A intron #1, soybean FAD3-1A intron #4 ends, soybean FATB intron #2 ends and soybean FAD3-1A intron #5).

Step 12—Soybean FAD3-1B intron #4 is PCR amplified using genomic DNA as template and the following primers:

5′ Primer=19516=CCCAAGCTTGGGGTATCCCATTTAACAC (Adding HindIII site to 5′ end)

3′ Primer=19515 GACCCGGGTCCTGTGAAATTACATATAGAC (Adding XmaCI site to 3′ end)

The resulting PCR product is cloned into PCR 2.1 to form KAWHIT03.0090.

Step 13—To add the soybean FAD3-1B intron #4 into KAWHIT03.0077, plasmids KAWHIT03.0090 and KAWHIT03.0077 are digested with HindIII and XmaCI and directionally ligated to make KAWHIT03.0091 (Soybean FAD2-1A intron#1, soybean FAD3-1A intron #4 ends, soybean FATB intron #2 ends, soybean FAD3-1A intron #4 and soybean FAD3-1A intron #5).

Step 14—KAWHIT03.0091 is digested with BstXI and SalI and the fragment containing the four introns (Soybean FAD2-1A intron #1, soybean FAD3-1A intron #4 ends, soybean FATB intron #2 ends, soybean FAD3-1A intron #4) is gel purified. In a different tube KAWHIT03.0091, is also digested with XhoI and Sse83871. The four intron fragment is then cloned back into KAWHIT03.0091 in the opposite orientation on the other site of Soy FAD3-1A intron #5 to create KAWHIT03.0092 (soybean FAD2-1A intron #1 sense, soybean FAD3-1A intron #4 ends sense, soybean FATB intron #2 ends sense, soybean FAD3-1A intron #4 sense, spliceable soybean FAD3-1A intron #5, soy FAD3-1B intron #4 anti-sense, soybean FATB intron #2 ends anti-sense, soybean FAD3-1A intron #4 ends anti-sense, soybean FAD2-1A intron #1 anti-sense).

Step 15—To link the RNAi construct to the 7S alpha′ promoter and the TML 3′, KAWHIT03.0092 and pMON68527 (7Sa′/TML3′ cassette) are digested with SacI and ligated together to make KAWHIT03.0093 0092 (7S alpha′ promoter—FAD2-1A intron #1 sense, soybean FAD3-1A intron #4 ends sense, soybean FATB intron #2 ends sense, soybean FAD3-1A intron #4 sense, spliceable soybean FAD3-1A Intron #5, soy FAD3-1B intron #4 anti-sense, soybean FATB intron #2 ends anti-sense, soybean FAD3-1A intron #4 ends anti-sense, soybean FAD2-1A intron #1 anti-sense—TML3′).

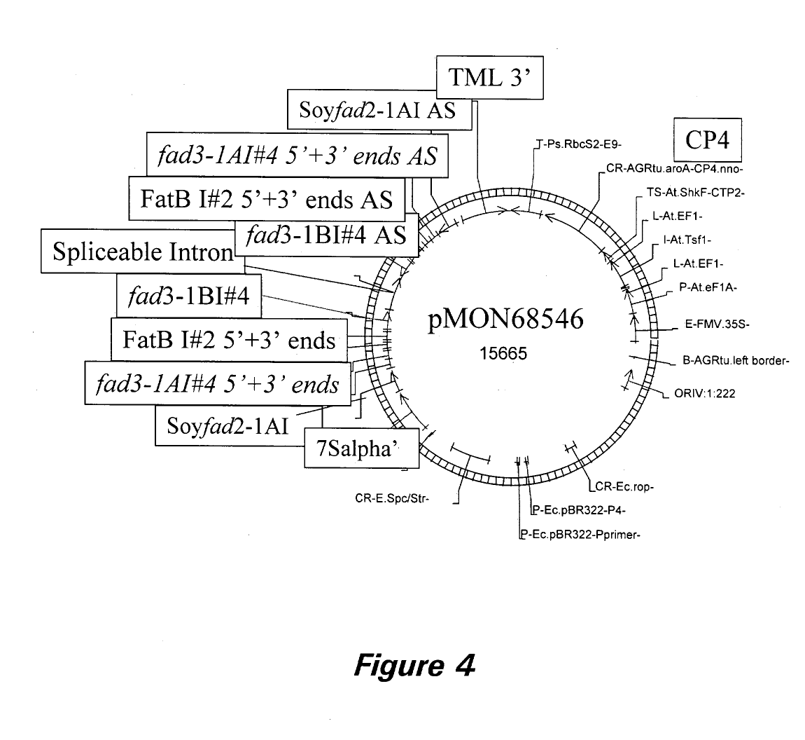

Step 16—To introduce the assembled RNAi construct into pMON80612, which contains the selectable maker CP4 fused to the FMV promoter and the RBCS 3′, KAWHIT03.0093 and pMON80612 are digested with NotI and ligated together to form pMON68456 (illustrated in FIG. 4) comprising a 7S alpha′ promoter operably linked to the intron series, double-stranded-RNA-forming construct of FAD2-1A intron #1 sense, soybean FAD3-1A intron #4 ends sense, soybean FATB intron #2 ends sense, soybean FAD3-1A intron #4 sense, spliceable soybean FAD3-1A intron #5, soy FAD3-1B intron #4 anti-sense, soybean FATB intron #2 ends anti-sense, soybean FAD3-1A intron #4 ends anti-sense, soybean FAD2-1A intron #1 anti-sense and TML3′ terminator).

Representative sequences for FAD2-1A, FAD2-1B, FAD2-2B, FAD3-1A, FAD3-1B, and FAD3-1C introns include, without limitation, those set forth in U.S. application Ser. No. 10/176,149, filed Jun. 21, 2002, and U.S. patent application Ser. No. 09/638,508, filed Aug. 11, 2000, and U.S. Provisional Application Serial No. 60/151,224, filed Aug. 26, 1999, and U.S. Provisional Application Serial No. 60/172,128, filed Dec. 17, 1999, all of which applications are herein incorporated by reference in their entireties including, without limitation, their accompanying sequence listings.

Representative sequences for FATB introns include, without limitation, those set forth in U.S. Provisional Application Serial No. 60/390,185, filed Jun. 21, 2002, which application is herein incorporated by reference in its entirety, including without limitation its sequence listing.

### Example 5

### This Example Illustrates the Preparation of a Variety of Intron dsRNA-Forming Constructs Which Can Suppress One or a Plurality of Genes in Soybean

Using the step-wise method illustrated in Example 4, intron dsRNA-forming vectors are constructed to have the following elements:

(1) 7S promoter—FAD2-1A sense intron—FAD3-1C sense intron—FAD3-1A sense intron—FAD3-1B sense intron—spliceable FAD3 intron #5—FAD3-1B anti-sense intron—FAD3-1A anti-sense intron—FAD3-1C anti-sense intron—FAD2-1A anti-sense intron—pea rbcS;

(2) 7S promoter—FAD2-1A sense intron—FAD3-1A sense intron—FAD3-1B sense intron—spliceable FAD3 intron #5—FAD3-1B anti-sense intron—FAD3-1A anti-sense intron—FAD2-1A anti-sense intron—pea rbcS;

(3) 7S promoter—FAD2-1A sense intron—FAD3-1A sense intron—spliceable FAD3 intron #5—FAD3-1A anti-sense intron—FAD2-1A anti-sense intron—pea rbcS;

(4) 7S promoter—FAD2-1A sense intron—spliceable FAD3 intron #5—FAD2-1A anti-sense intron—pea rbcS;

(5) 7S promoter—FAD3-1A sense intron—spliceable FAD3 intron #5—FAD3-1A anti-sense intron—pea rbcS;

(6) 7S promoter—FAD2-1A sense intron—FAD3-1A sense 3′UTR—spliceable FAD3 intron #5—FAD3-1A anti-sense 3′UTR—FAD2-1A anti-sense intron—pea rbcS; and

(7) 7S promoter—FAD2-1A sense intron—FAD3-1A sense 3′UTR—FAD3-1B sense 3′UTR—spliceable FAD3 intron #5—FAD3-1B anti-sense 3′UTR—FAD3-1A anti-sense 3′UTR—FAD2-1A anti-sense intron—pea rbcS;

(8) 7S promoter—FATB sense intron I—FATB sense intron II—spliceable FAD3 intron #5—FATB anti-sense intron II—FATB anti-sense intron I—pea rbcS;

(9) 7S promoter—FATB sense intron II—FATB sense intron I—spliceable FAD3 intron #5—FATB anti-sense intron I—FATB anti-sense intron II—pea rbcS;

(10) 7S promoter—FATB sense intron—spliceable FAD3 intron #5—FATB anti-sense intron—pea rbcS;

(11) 7S promoter—FAD2-1A sense intron—FAD3-1C sense intron—FAD3-1A sense intron—FAD3-1B sense intron—FATB sense intron—spliceable FAD3 intron #5—FATB anti-sense intron—FAD3-1B anti-sense intron—FAD3-1A anti-sense intron—FAD3-1C anti-sense intron—FAD2-1A anti-sense intron—pea rbcS;

(12) 7S promoter—FAD2-1A sense intron—FAD3-1A sense intron—FAD3-1B sense intron—FATB sense intron—spliceable FAD3 intron #5—FATB anti-sense intron—FAD3-1B anti-sense intron—FAD3-1A anti-sense intron—FAD2-1A anti-sense intron—pea rbcS; and

(13) 7S promoter—FAD2-1A sense intron sense intron—FAD3-1A sense intron—FATB sense intron—spliceable FAD3 intron #5—FATB anti-sense intron—FAD3-1A anti-sense intron—FAD2-1A anti-sense intron—pea rbcS.

### Example 6

### This Example Illustrates Plant Transformation with the Constructs of this Invention to Produce Soybean Plants with Suppressed Genes

A transformation vector pMON68456 as prepared in Example 4 is used to introduce an intron double-stranded RNA-forming construct into soybean for suppressing the A12 desaturase, A 15 desaturase, and FA TB genes. The vector is stably introduced into soybean (Asgrow variety A4922) via Agrobacterium tumefaciens strain ABI (Martinell, U.S. Pat. No. 6,384,301). The CP4 selectable marker allows transformed soybean plants to be identified by selection on media containing glyphosate herbicide.

Fatty acid compositions are analyzed from seed of soybean lines transformed with the intron expression constructs using gas chromatography. R₁ pooled seed and R₁ single seed oil compositions demonstrate that the mono- and polyunsaturated fatty acid compositions were altered in the oil of seeds from transgenic soybean lines as compared to that of the seed from non-transformed soybean. For instance, FAD2 suppression provides plants with increased amount of oleic acid ester compounds; FAD3 suppression provides plants with decreased linolenic acid ester compounds; and FATB suppression provides plants with reduced saturated fatty ester compounds, e.g. palmitates and stearates. Selections can be made from such lines depending on the desired relative fatty acid composition. Fatty acid compositions are analyzed from seed of soybean lines transformed with constructs using gas chromatography.

### Example 7

### This Example Illustrates Transient Expression Of Constructs for Intron Double-Stranded RNA Gene Suppression

DNA containing the expression constructs for sense, antisense, and dsRNA expression of the Δ12 desaturase, Δ15 desaturase, and FATB introns is transferred into the nucleus or the cytoplasm of tobacco mesophyll protoplasts. The DNA constructs illustrated in Examples 3, 4, 5 and are introduced by microinjection as described (Crossway et al., (1986) Mol. Gen. Genet. 202: 179-185). Transient gene suppression is observed, e.g., by measuring RNA or fatty acid compound compositions.

All publications and patent applications mentioned in this specification are herein incorporated by reference to the same extent as if each individual publication or patent application was specifically and individually indicated to be incorporated by reference.

What is claimed is:

1. A nucleic acid construct comprising DNA which is transcribed into RNA that forms at least one double-stranded RNA molecule, wherein one strand of said double-stranded molecule is coded by a portion of said DNA which is at least 90% identical to at least one transcribed intron of a gene.

2. The construct of claim 1, wherein one strand of said double-stranded molecule is coded by a portion of said DNA which is at least 98% identical to at least one transcribed intron of a gene.

3. The construct of claim 1, wherein one strand of said double-stranded molecule is coded by a portion of said DNA which is 100% identical to at least one transcribed intron of a gene.

4. The construct of claim 1, comprising in series one strand of an intron, a spliceable intron, and the complement of said intron, wherein said spliceable intron provides a hairpin structure, and wherein said intron and said complement of said intron can hybridize to each other.

5. The construct of claim 1, wherein said transcribed introns are in FAD2 genes or FAD3 genes.

6. The construct of claim 1, comprising DNA which is transcribed into RNA that forms at least one double-stranded RNA molecule wherein one strand of said double-stranded molecule is coded by a portion of said DNA which is at least 90% identical to at least two transcribed introns.

7. The construct of claim 6, comprising DNA which is transcribed into RNA that forms two or more double-stranded RNA molecules.

8. A transformed cell or organism having in its genome an introduced nucleic acid construct comprising DNA which is transcribed into RNA that forms at least one double-stranded RNA molecule, wherein one strand of said double-stranded molecule is coded by a portion of said DNA which is at least 90% identical to at least one transcribed intron of a gene.

9. A transformed plant having in its genome an introduced nucleic acid construct comprising DNA which is transcribed into RNA that forms at least one double-stranded RNA molecule, wherein one strand of said double-stranded molecule is coded by a portion of said DNA which is at least 90% identical to at least one transcribed intron of a gene.

10. The transformed plant of claim 9, having in its genome an introduced nucleic acid construct comprising DNA which is transcribed into RNA that forms at least one double-stranded RNA molecule wherein one strand of said double-stranded molecule is coded by a portion of said DNA which is at least 98% identical to at least one transcribed intron of a native plant gene.

11. The transformed plant of claim 9, wherein said intron is from a FAD2 gene or a FAD3 gene.

12. The transformed plant of claim 11, wherein expression of a protein encoded by said FAD2 gene or said FAD3 gene is reduced.

13. The transformed plant of claim 11, wherein expression of a protein encoded by said FAD2 gene or said FAD3 gene is substantially reduced.

14. The transformed plant of claim 11, wherein expression of the protein encoded by said FAD2 gene or said FAD3 gene is effectively eliminated.

15. A method of reducing expression of a protein encoded by a target gene in a mammal comprising introducing into a cell or organism a nucleic acid construct comprising DNA which is transcribed into RNA that forms at least one double-stranded RNA molecule, wherein one strand of said double-stranded molecule is coded by a portion of said DNA which is at least 90% identical to at least one transcribed intron of a gene.

16. The method of claim 15, wherein the target gene encodes a protein in an insect or nematode which is a pest to a plant, and wherein said method comprises introducing into the genome of said plant a nucleic acid construct comprising DNA which is transcribed into RNA that forms at least one double-stranded RNA molecule which is effective for reducing expression of said target gene when said insect or nematode ingests cells from said plant.

17. A method of reducing expression of a protein encoded by a target gene in a plant comprising introducing into a plant genome a nucleic acid construct comprising DNA which is transcribed into RNA that forms at least one double-stranded RNA molecule, wherein one strand of said double-stranded molecule is coded by a portion of said DNA which is at least 90% identical to at least one transcribed intron of a gene.


SEQUENCE LISTING (55 sequences)

SEQ ID NO: 1 - DNA, 420 bp, Glycine max
gtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtcatgatttcactctctctaatctctccattcattttgtagttgtcattatctttagatttttcactacctggtttaaaattgagggattgtagttctgttggtacatattacacattcagcaaaacaactgaaactcaactgaacttgtttatactttgacacag

SEQ ID NO: 2 - DNA, 405 bp, Glycine max
gtatgatgctaaattaaattgtgcctgcaccccaggatatttcatgtgggattcatcatttattgaggaaaactctccaaattgaatcgtgcatttatattttttttccatttctagatttcttgaaggcttatggtataggcacctacaattatcagcacttctctctattgataaacaattggctgtaataccacagtagagaacgatcacaacattttgtgctggttaccttttgttttatggtcatgatttcactctctctaatctgtcacttccctccattcattttgtacttctcatatttttcacttcctggttgaaaattgtagttctcttggtacatactagtattagacattcagcaacaacaactgaactgaacttctttatactttgacacag

SEQ ID NO: 3 - DNA, 6220 bp, Glycine max
agcttggtaccgagctcggatccactagtaacggccgccagtgtgctggaattcggcttctctctcaccctcctcttcacacattttctgtgcgctctaacaaacattctcgttcacactttcaggtacttttctctccttatctctttatctttattctttcctactttattgcttaaaccaatgctatctatgcttcgatctcgccttcttattttccacttcccttttctcgcttgatctaaccgttttcgccctccgcgcttcgattgactgagtacatctacgattctctgttctttcatttcatagatttcgtctgattttggctaacttggtttctgttgcggccgattcttacatatactgattgtttagcataaatgaacttgcttgtttagcactatctgcatattttcgtcacgcatctctttcggatctaaggatgaatctcctatttcctccgtattatttctcgtatctcttgttctgtgctaatgctccagaaaatggcagcattgtcttcttctttgctgtataagtgtttgtgttgtgaatctggaagcgattttgcgtgaggtaacttgcgacttcaactattatctttcagatctcgttaatttattagctgctattaatttgtgtgtgcagtgtcaaactgaagcacacgactgcttagaagttagaatttgactgactgttcctctttgatttttttctttcttttctttgctwactcggcctatttaatgatctttataaatagattagtggaccacttggttagttggtgagttatgaatattcgaattttctaccacaagttgggttaaaaaaatctctgcaactacacgaggattttttattttatttagaggaaactattctgtcatcctttttccgattacacttttctatcagttgttttgaaatatacaccttaggaatataatattacccctttcggtcttaatataaatatattttaattatttatattttatttaatgaaattatttttaaaatactttcatttaatagaatttttaataaagttaaagacttttattgtgtagagtttaacgaagttaattagttttcttagtaaatgtaaaatatgccttttttgttgtttataatggagattggaaaaaatatactttaatttttttcaagtgatgaataattatggatgttttgtcaatatttttgtcttgctatacaactttcagtcttgccattaaataattttgaatgtgttattgatatctctgaacaatatttagagacgaacataaattttatatattttatataatttctttttattacccttttattatcaattttgaaatttggttaatatctgtgtttcattttgaggtctcaaatttgatataaggaggttcaaaatgcgttgctagccattttaaagattagcaggagaggaaatgtttctggacttaaatttaaaatatgcttatttgtttttcaagagagagagatcaatatttatataatacacttgaattaatatacaccattgttgcaaaaaaaaaaaaatattagttgattgtgtgacaatattttatattaaatataattagttaatttagttcaagttgagttacatttttacataccattcttagccgccacttttttatatttatttgtaggaataacttttcatctgtatcaattttccccgtctaataaaaagggtttgactttttcttataatagagtttttttttttttgctttaagttattgtaaaataattattttattttttttgcctttgtaaattatgtatatttaatgttttaataggaaaaaaatgttatcaaaagcactaaaagactaaaattaaacaaccataatttgcaaagatgaaaataaaaaaataattttgtaaagataaaaaatgaaataaaatagttaaattataggaatttaaaagctatttaaatcaacaaaagttaaagtttctgtaaaaaaagttcaatttttttttttattattgaaaaagttaaagctaatgagcgttcgatttgggttagtatgtagtatttattattttcaagattttggattttattgtcgatgtttctgatttgaatataattattttccattcaacttgtgattttataagaaaaaaaaaggtacagaaaaaatcaagcgctttttttatttcaattagtggaggtttcactgaaatgggtaaagaatctattttgcaatcacaattattaccggtattcaactgcaacaaggaacaaaattcctttcgtaaatatacggagaggaatctattttgacttgttgaatttatggtaaagtagaatttagaatttaattatgagttgaagtaattttgaataatttatatgttaaatataaaattttgtactaagttttattcataactttgattctataatacaaacatacataagttcaaaaataattttaattaaaattaattttatcaatttttattcaaacacgagtctaatttgcttgatgaattaagaaaataaggaagaaaatattaaaaactaggagagaagttaaagagaatttcatctttattattctcagttgtttcaaaaataatgaaaggatagctatataatactgtaactgagccaagaacatatttgccgtccgagtaaccttttcttttcttgttccgttttctccgccgatgaagagagggaagggaatgtatctttgtatttatgttttcaaagagttcgtgcataaaattggtttaatcaaatttttcataagattattattttatgattttttaaaataaattagtaactatattccgtaagtcgtacacagttatatgtagtaagtaaattatattttaataattattatcttaaaattttcttaagaacttggttaaaatatttttgtttgaaaaagtttatgataacttttttttgttgaaaaaaagtttacgattatctaactcgtacttagattatttctaattgggatttattgaagggttttttaagtaaagaaattgtttcttatggtttcttttttattggacaaatttacgtagcaaagagtgtttcttaaaaacaagacatgtatcctttgaaaaaaaactatttctttgaaataaaaaataatatttatctggcacataataatgttaaaattaaatcataattaggtaaaaataaaataaatataaaagtatgagtttgttaagttttttataattttttattattaaagtaaaattatgtatgatttttttataatgatatgatattttagggatcacaaaaaataatgtggtgaatacaaaagtaactcaaaaaattcatttagtaaattttcattggagatgctattattatgctttctgattgctttgtccaaaaaataaagaatgtttttttatttgaaaattgaaaatttctgggtcatgttaagatcttgtagacggtaacgtcggcctaaagttgtgtgaggggtgttgcatgcaccgatcattaattactcgatatggaaaacgactgaaataatttaatttgatgttgctaatattggccatccctctcatcattattgtttttttatttgtaacatgacatattcttgtgggtccgctacggattgggtgtttgttgccaaaaaatacaaaatatctgtggaacaaggataaacagtcttgtttgtttaattgattgattgatgagtttgcaagctatatttttaatttattttaattaaacttttgtgttttagttctacaattttattcatcttgattttttttttacttggcaaaatcatgattttttaatttttacttatgttgaaaacaaatttattgctaaaaaaacatttattctttttttagagaaaaaacaaatttgtgatatgtagtgaatcaaatgaaaattttaaacataatatagaatactctacaaatcaattttgagtttctttatcattttatttatttattgacatacttctactttctgcaaagaccctgactcgtggaagatatagggaaggttatggaagttagtgtattgtcatatctagctatctttgctaattgaaaaagccttccctttgtttacagatctggataaggttgcatgtttattcttttcaactgtgaatggttctttgcatcttttttagtatatgagattaatgttttaattaggaagaagcttttagaacatcacccgaatccaattcgttttggtttctgtgatcttgatgtaaatctatactaatttggtttgggcagaagaaaatgttctttgctcaagtcctctaggacgaaaatataaatataacagggtatatcagatctctattcttctgtgggtaatgatagcatgtttctgttgttttcttattcttcattggtcatgataacctgctaattctatttgccacgattgagatgaaaaggtaatgaactagtaaacaataatgagaagaatatgtcgctactattgttgaaacggttacgccaggcacttgagtatgatgcactattttaattaatgcattttttttgctttgatgagaacgcacattgttcattctgattcggtgagtttagaaactattgctgataatccttgatttaagattttagtcttgttcatgttcattaaaagtgttgtaaaaaaatgcactgatatgtcatgtgcagattgtgtgaagatgggggcgggtggccgaactgatgttcctcctgccaacaggaagtcagaggttgaccctttgaagcgggtgccatttgaaaaacctccatttagtctcagccaaatcaagaaggtcattccacctcactgtttccagcgttctgttttccgctcattctcctatgttgtttacgacctcaccatagccttctgcctctattatgttgccacccattacttccacctccttcccagccctctctctttcttggcatggccaatctactgggctgtccaaggttgcatccttactggagtttgggtcattgcccatgagtgtggccaccatgcattcagtgactaccagttgcttgatgatattgttggccttgtcctccactccggtctcctagtcccatacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcagtatgctatggccttttccgtcttgccatggcaaaaggacttgcctgggtggtgtgtgtttatggagttccattgctagtggtcaatggatttttggtgttgattacattcttgcagcatactcaccctgcattgccacattacacttcctctgagtgggactggttgagaggagctttagcaacagtggatagagattatggaatcctgaacaaggtcttccataatattacagacactcatgtagcacatcacttgttctccacaatgccacattatcatgcaatggaggctacaaaggcaataaaacccattttgggagagtattatcggtttgatgagactccatttgtcaaggcaatgtggagagaggcaagagagtgtatttatgtggagccagatcaaagtaccgagagcaaaggtgtattttggtacaacaataagttgtgatgattaatgtagccgaggcttctttgaactttcccttgtgactgtttagtatcatggttgcttattgggaataattttgttgaaccctgatgttggtagtaagtatctagacagttgcatagcggttttgtttacagaataagatatagcctctctgaacagtttgattattgcaccatggtttgcaatcggtgcatgtcgaccaagtttctcaagactgtggagaagcttattcttgttccagttcttgaatccaagttgttaccgtattctgtaagccgaattctgcagatatccatcacactggcggccgctcgagcatgcatctagagggc

SEQ ID NO: 4 - DNA, 4597 bp, Glycine max
gtacttttctctccttatctctttatctttattctttcctactttattgcttaaaccaatgctatctatgcttcgatctcgccttcttattttccacttcccttttctcgcttgatctaaccgttttcgccctccgcgcttcgattgactgagtacatctacgattctctgttctttcatttcatagatttcgtctgattttggctaacttggtttctgttgcggccgattcttacatatactgattgtttagcataaatgaacttgcttgtttagcactatctgcatattttcgtcacgcatctctttcggatctaaggatgaatctcctatttcctccgtattatttctcgtatctcttgttctgtgctaatgctccagaaaatggcagcattgtcttcttctttgctgtataagtgtttgtgttgtgaatctggaagcgattttgcgtgaggtaacttgcgacttcaactattatctttcagatctcgttaatttattagctgctattaatttgtgtgtgcagtgtcaaactgaagcacacgactgcttagaagttagaatttgactgactgttcctctttgatttttttctttcttttctttgctwactcggcctatttaatgatctttataaatagattagtggaccacttggttagttggtgagttatgaatattcgaattttctaccacaagttgggttaaaaaaatctctgcaactacacgaggattttttattttatttagaggaaactattctgtcatcctttttccgattacacttttctatcagttgttttgaaatatacaccttaggaatataatattacccctttcggtcttaatataaatatattttaattatttatattttatttaatgaaattatttttaaaatactttcatttaatagaatttttaataaagttaaagacttttattgtgtagagtttaacgaagttaattagttttcttagtaaatgtaaaatatgccttttttgttgtttataatggagattggaaaaaatatactttaatttttttcaagtgatgaataattatggatgttttgtcaatatttttgtcttgctatacaactttcagtcttgccattaaataattttgaatgtgttattgatatctctgaacaatatttagagacgaacataaattttatatattttatataatttctttttattacccttttattatcaattttgaaatttggttaatatctgtgtttcattttgaggtctcaaatttgatataaggaggttcaaaatgcgttgctagccattttaaagattagcaggagaggaaatgtttctggacttaaatttaaaatatgcttatttgtttttcaagagagagagatcaatatttatataatacacttgaattaatatacaccattgttgcaaaaaaaaaaaaatattagttgattgtgtgacaatattttatattaaatataattagttaatttagttcaagttgagttacatttttacataccattcttagccgccacttttttatatttatttgtaggaataacttttcatctgtatcaattttccccgtctaataaaaagggtttgactttttcttataatagagtttttttttttttgctttaagttattgtaaaataattattttattttttttgcctttgtaaattatgtatatttaatgttttaataggaaaaaaatgttatcaaaagcactaaaagactaaaattaaacaaccataatttgcaaagatgaaaataaaaaaataattttgtaaagataaaaaatgaaataaaatagttaaattataggaatttaaaagctatttaaatcaacaaaagttaaagtttctgtaaaaaaagttcaatttttttttttattattgaaaaagttaaagctaatgagcgttcgatttgggttagtatgtagtatttattattttcaagattttggattttattgtcgatgtttctgatttgaatataattattttccattcaacttgtgattttataagaaaaaaaaaggtacagaaaaaatcaagcgctttttttatttcaattagtggaggtttcactgaaatgggtaaagaatctattttgcaatcacaattattaccggtattcaactgcaacaaggaacaaaattcctttcgtaaatatacggagaggaatctattttgacttgttgaatttatggtaaagtagaatttagaatttaattatgagttgaagtaattttgaataatttatatgttaaatataaaattttgtactaagttttattcataactttgattctataatacaaacatacataagttcaaaaataattttaattaaaattaattttatcaatttttattcaaacacgagtctaatttgcttgatgaattaagaaaataaggaagaaaatattaaaaactaggagagaagttaaagagaatttcatctttattattctcagttgtttcaaaaataatgaaaggatagctatataatactgtaactgagccaagaacatatttgccgtccgagtaaccttttcttttcttgttccgttttctccgccgatgaagagagggaagggaatgtatctttgtatttatgttttcaaagagttcgtgcataaaattggtttaatcaaatttttcataagattattattttatgattttttaaaataaattagtaactatattccgtaagtcgtacacagttatatgtagtaagtaaattatattttaataattattatcttaaaattttcttaagaacttggttaaaatatttttgtttgaaaaagtttatgataacttttttttgttgaaaaaaagtttacgattatctaactcgtacttagattatttctaattgggatttattgaagggttttttaagtaaagaaattgtttcttatggtttcttttttattggacaaatttacgtagcaaagagtgtttcttaaaaacaagacatgtatcctttgaaaaaaaactatttctttgaaataaaaaataatatttatctggcacataataatgttaaaattaaatcataattaggtaaaaataaaataaatataaaagtatgagtttgttaagttttttataattttttattattaaagtaaaattatgtatgatttttttataatgatatgatattttagggatcacaaaaaataatgtggtgaatacaaaagtaactcaaaaaattcatttagtaaattttcattggagatgctattattatgctttctgattgctttgtccaaaaaataaagaatgtttttttatttgaaaattgaaaatttctgggtcatgttaagatcttgtagacggtaacgtcggcctaaagttgtgtgaggggtgttgcatgcaccgatcattaattactcgatatggaaaacgactgaaataatttaatttgatgttgctaatattggccatccctctcatcattattgtttttttatttgtaacatgacatattcttgtgggtccgctacggattgggtgtttgttgccaaaaaatacaaaatatctgtggaacaaggataaacagtcttgtttgtttaattgattgattgatgagtttgcaagctatatttttaatttattttaattaaacttttgtgttttagttctacaattttattcatcttgattttttttttacttggcaaaatcatgattttttaatttttacttatgttgaaaacaaatttattgctaaaaaaacatttattctttttttagagaaaaaacaaatttgtgatatgtagtgaatcaaatgaaaattttaaacataatatagaatactctacaaatcaattttgagtttctttatcattttatttatttattgacatacttctactttctgcaaagaccctgactcgtggaagatatagggaaggttatggaagttagtgtattgtcatatctagctatctttgctaattgaaaaagccttccctttgtttacagatctggataaggttgcatgtttattcttttcaactgtgaatggttctttgcatcttttttagtatatgagattaatgttttaattaggaagaagcttttagaacatcacccgaatccaattcgttttggtttctgtgatcttgatgtaaatctatactaatttggtttgggcagaagaaaatgttctttgctcaagtcctctaggacgaaaatataaatataacagggtatatcagatctctattcttctgtgggtaatgatagcatgtttctgttgttttcttattcttcattggtcatgataacctgctaattctatttgccacgattgagatgaaaaggtaatgaactagtaaacaataatgagaagaatatgtcgctactattgttgaaacggttacgccaggcacttgagtatgatgcactattttaattaatgcattttttttgctttgatgagaacgcacattgttcattctgattcggtgagtttagaaactattgctgataatccttgatttaagattttagtcttgttcatgttcattaaaagtgttgtaaaaaaatgcactgatatgtcatgtgcag

SEQ ID NO: 5 - DNA, 191 bp, Glycine max
gtaataatttttgtgtttcttactcttttttttttttttttgtttatgatatgaatctcacacattgttctgttatgtcatttcttcttcatttggctttagacaacttaaatttgagatctttattatgtttttgcttatatggtaaagtgattcttcattatttcattcttcattgattgaattgaaca

SEQ ID NO: 6 - DNA, 346 bp, Glycine max
ttagttcatactggcttttttgtttgttcatttgtcattgaaaaaaaatcttttgttgattcaattatttttatagtgtgtttggaagcccgtttgagaaaataagaaatcgcatctggaatgtgaaagttataactatttagcttcatctgtcgttgcaagttcttttattggttaaatttttatagcgtgctaggaaacccattcgagaaaataagaaatcacatctggaatgtgaaagttataactgttagcttctgagtaaacgtggaaaaaccacattttggatttggaaccaaattttatttgataaatgacaaccaaattgattttgatggattttgca

SEQ ID NO: 7 - DNA, 142 bp, Glycine max
gtatgtgattaattgcttctcctatagttgttcttgattcaattacattttatttatttggtaggtccaagaaaaaagggaatctttatgcttcctgaggctgttcttgaacatggctcttttttatgtgtcattatcttag

SEQ ID NO: 8 - DNA, 1228 bp, Glycine max
taacaaaaataaatagaaaatagtgggtgaacacttaaatgcgagatagtaatacctaaaaaaagaaaaaaatataggtataataaataatataactttcaaaataaaaagaaatcatagagtctagcgtagtgtttggagtgaaatgatgttcacctaccattactcaaagattttgttgtgtcccttagttcattcttattattttacatatcttacttgaaaagactttttaattattcattgagatcttaaagtgactgttaaattaaaataaaaaacaagtttgttaaaacttcaaataaataagagtgaagggagtgtcatttgtcttctttcttttattgcgttattaatcacgtttctcttctcttttttttttttcttctctgctttccacccattatcaagttcatgtgaagcagtggcggatctatgtaaatgagtggggggcaattgcacccacaagattttattttttatttgtacaggaataataaaataaaactttgcccccataaaaaataaatattttttcttaaaataatgcaaaataaatataagaaataaaaagagaataaattattattaattttattattttgtactttttatttagtttttttagcggttagatttttttttcatgacattatgtaatcttttaaaagcatgtaatatttttattttgtgaaaataaatataaatgatcatattagtctcagaatgtataaactaataataattttatcactaaaagaaattctaatttagtccataaataagtaaaacaagtgacaattatattttatatttacttaatgtgaaataatacttgaacattataataaaacttaatgacaggagatattacatagtgccataaagatattttaaaaaataaaatcattaatacactgtactactatataatattcgatatatatttttaacatgattctcaatagaaaaattgtattgattatattttattagacatgaatttacaagccccgtttttcatttatagctcttacctgtgatctattgttttgcttcgctgtttttgttggtcaagggacttagatgtcacaatattaatactagaagtaaatatttatgaaaacatgtaccttacctcaacaaagaaagtgtggtaagtggcaacacacgtgttgcatttttggcccagcaataacacgtgtttttgtggtgtactaaaatggac

SEQ ID NO: 9 - DNA, 625 bp, Glycine max
gtacattttattgcttattcacctaaaaacaatacaattagtacatttgttttatctcttggaagttagtcattttcagttgcatgattctaatgctctctccattcttaaatcatgttttcacacccacttcatttaaaataagaacgtgggtgttattttaatttctattcactaacatgagaaattaacttatttcaagtaataattttaaaatatttttatgctattattttattacaaataattatgtatattaagtttattgattttataataattatattaaaattatatcgatattaatttttgattcactgatagtgttttatattgttagtactgtgcatttattttaaaattggcataaataatatatgtaaccagctcactatactatactgggagcttggtggtgaaaggggttcccaaccctcctttctaggtgtacatgctttgatacttctggtaccttcttatatcaatataaattatattttgctgataaaaaaacatggttaaccattaaattctttttttaaaaaaaaaactgtatctaaactttgtattattaaaaagaagtctgagattaacaataaactaacactcatttggattcactgca

SEQ ID NO: 10 - DNA, 98 bp, Glycine max
ggtgagtgattttttgacttggaagacaacaacacattattattataatatggttcaaaacaatgactttttctttatgatgtgaactccatttttta

SEQ ID NO: 11 - DNA, 115 bp, Glycine max
ggtaactaaattactcctacattgttactttttcctccttttttttattatttcaattctccaattggaaatttgaaatagttaccataattatgtaattgtttgatcatgtgca

SEQ ID NO: 12 - DNA, 148 bp, Glycine max
  FAD3-1B intron 3c
gtaatctcactctcacactttctttatacatcgcacgccagtgtgggttatttgcaacctacaccgaagtaatgccctataattaatgaggttaacacatgtccaagtccaatattttgttcacttatttgaacttgaacatgtgtag

SEQ ID NO: 13 - DNA, 361 bp, Glycine max
  FAD3-1B intron 4
gtatcccatttaacacaatttgtttcattaacattttaagagaatttttttttcaaaatagttttcgaaattaagcaaataccaagcaaattgttagatctacgcttgtacttgttttaaagtcaaattcatgaccaaattgtcctcacaagtccaaaccgtccactattttattttcacctactttatagcccaatttgccatttggttacttcagaaaagagaaccccatttgtagtaaatatattatttatgaattatggtagtttcaacataaaacatacttatgtgcagttttgccatccttcaaaagaaggtagaaacttactccatgttactctgtctatatgtaatttcacag

SEQ ID NO: 14 - DNA, 1037 bp, Glycine max
gtaacaaaaataaatagaaaatagtgagtgaacacttaaatgttagatactaccttcttcttctttttttttttttttttgaggttaatgctagataatagctagaaagagaaagaaagacaaatataggtaaaaataaataatataacctgggaagaagaaaacataaaaaaagaaataatagagtctacgtaatgtttggatttttgagtgaaatggtgttcacctaccattactcaaagattctgttgtctacgtagtgtttggactttggagtgaaatggtgttcacctaccattactcagattctgttgtgtcccttagttactgtcttatattcttagggtatattctttattttacatccttttcacatcttacttgaaaagattttaattattcattgaaatattaacgtgacagttaaattaaaataataaaaaattcgttaaaacttcaaataaataagagtgaaaggatcatcatttttcttctttcttttattgcgttattaatcatgcttctcttcttttttttcttcgctttccacccatatcaaattcatgtgaagtatgagaaaatcacgattcaatggaaagctacaggaacyttttttgttttgtttttataatcggaattaatttatactccattttttcacaataaatgttacttagtgccttaaagataatatttgaaaaattaaaaaaattattaatacactgtactactatataatatttgacatatatttaacatgattttctattgaaaatttgtatttattattttttaatcaaaacccataaggcattaatttacaagacccatttttcatttatagctttacctgtgatcatttatagctttaagggacttagatgttacaatcttaattacaagtaaatatttatgaaaaacatgtgtcttaccccttaaccttacctcaacaaagaaagtgtgataagtggcaacacacgtgttgcttttttggcccagcaataacacgtgtttttgtggtgtacaaaaatggacag

SEQ ID NO: 15 - DNA, 4497 bp, Glycine max
cttgcttggtaacaacgtcgtcaagttattattttgttctttttttttttatcatatttcttattttgttccaagtatgtcatattttgatccatcttgacaagtagattgtcatgtaggaataggaatatcactttaaattttaaagcattgattagtctgtaggcaatattgtcttcttcttcctccttattaatattttttattctgccttcaatcaccagttatgggagatggatgtaatactaaataccatagttgttctgcttgaagtttagttgtatagttgttctgcttgaagtttagttgtgtgtaatgtttcagcgttggcttcccctgtaactgctacaatggtactgaatatatattttttgcattgttcatttttttcttttacttaatcttcattgctttgaaattaataaaacaaaaagaaggaccgaatagtttgaagtttgaactattgcctattcatgtaacttattcacccaatcttatatagtttttctggtagagatcattttaaattgaaggatataaattaagaggaaatacttgtatgtgatgtgtggcaatttggaagatcatgcgtagagagtttaatggcaggttttgcaaattgacctgtagtcataattacactgggccctctcggagttttgtgcctttttgttgtcgctgtgtttggttctgcatgttagcctcacacagatatttagtagttgttgttctgcatataagcctcacacgtatactaaacgagtgaacctcaaaatcatggccttacacctattgagtgaaattaatgaacagtgcatgtgagtatgtgactgtgacacaacccccggttttcatattgcaatgtgctactgtggtgattaaccttgctacactgtcgtccttgtttgtttccttatgtatattgataccataaattattactagtatatcattttatattgtccataccattacgtgtttatagtctctttatgacatgtaattgaattttttaattataaaaaataataaaacttaattacgtactataaagagatgctcttgactagaattgtgatctcctagtttcctaaccatatactaatatttgcttgtattgatagcccctccgttcccaagagtataaaactgcatcgaataatacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtcatgatttcactctctctaatctctccattcattttgtagttgtcattatctttagatttttcactacctggtttaaaattgagggattgtagttctgttggtacatattacacattcagcaaaacaactgaaactcaactgaacttgtttatactttgacacagggtctagcaaaggaaacaacaatgggaggtagaggtcgtgtggcaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattgatggagcaaccaatgggccatagtgggagttatggaagttttgtcatgtattagtacataattagtagaatgttataaataagtggatttgccgcgtaatgactttgtgtgtattgtgaaacagcttgttgcgatcatggttataatgtaaaaataattctggtattaattacatgtggaaagtgttctgcttatagctttctgcctaaaatgcacgctgcacgggacaatatcattggtaatttttttaaaatctgaattgaggctactcataatactatccataggacatcaaagacatgttgcattgactttaagcagaggttcatctagaggattactgcataggcttgaactacaagtaatttaagggacgagagcaactttagctctaccacgtcgttttacaaggttattaaaatcaaattgatcttattaaaactgaaaatttgtaataaaatgctattgaaaaattaaaatatagcaaacacctaaattggactgatttttagattcaaatttaataattaatctaaattaaacttaaattttataatatatgtcttgtaatatatcaagtttttttttttattattgagtttggaaacatataataaggaacattagttaatattgataatccactaagatcgacttagtattacagtatttggatgatttgtatgagatattcaaacttcactcttatcataatagagacaaaagttaatactgatggtggagaaaaaaaaatgttattgggagcatatggtaagataagacggataaaaatatgctgcagcctggagagctaatgtattttttggtgaagttttcaagtgacaactattcatgatgagaacacaataatattttctacttacctatcccacataaaatactgattttaataatgatgataaataatgattaaaatatttgattctttgttaagagaaataaggaaaacataaatattctcatggaaaaatcagcttgtaggagtagaaactttctgattataattttaatcaagtttaattcattcttttaattttattattagtacaaaatcattctcttgaatttagagatgtatgttgtagcttaatagtaattttttatttttataataaaattcaagcagtcaaatttcatccaaataatcgtgttcgtgggtgtaagtcagttattccttcttatcttaatatacacgcaaaggaaaaaataaaaataaaattcgaggaagcgcagcagcagctgataccacgttggttgacgaaactgataaaaagcgctgtcattgtgtctttgtttgatcatcttcacaatcacatctccagaacacaaagaagagtgacccttcttcttgttattccacttgcgttaggtttctactttcttctctctctctctctctctcttcattcctcatttttccctcaaacaatcaatcaattttcattcagattcgtaaatttctcgattagatcacggggttaggtctcccactttatcttttcccaagcctttctctttccccctttccctgtctgccccataaaattcaggatcggaaacgaactgggttcttgaatttcactctagattttgacaaattcgaagtgtgcatgcactgatgcgacccactcccccttttttgcattaaacaattatgaattgaggtttttcttgcgatcatcattgcttgaattgaatcatattaggtttagattct

SEQ ID NO: 16 - DNA, 18 bp, Artificial sequence
  PCR primer
atacaagccactaggcat

SEQ ID NO: 17 - DNA, 26 bp, Artificial sequence
  PCR primer
gattggccatgcaatgagggaaaagg

SEQ ID NO: 18 - DNA, 778 bp, Artificial sequence
  misc_feature (1)..(778): unsure at all n locations
atacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtcatgatttcactctctctaatctctccattcattttgtagttgtcattatctttagatttttcactacctggtttaaaattgagggattgtagttctgttggtacatattacacattcagcaaaacaactgaaactcaactgaacttgtttatactttgacacagggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatcaagccgaattctgcagatatccatcacatggcggcggntggngnaggnntntanagggcccaattc

SEQ ID NO: 19 - DNA, 2463 bp, Glycine max
actatagggcacgcgtggtcgacggcccgggctggtcctcggtgtgactcagccccaagtgacgccaaccaaacgcgtcctaactaaggtgtagaagaaacagatagtatataagtataccatataagaggagagtgagtggagaagcacttctcctttttttttctctgttgaaattgaaagtgttttccgggaaataaataaaataaattaaaatcttacacactctaggtaggtacttctaatttaatccacactttgactctatatatgttttaaaaataattataatgcgtacttacttcctcattatactaaatttaacatcgatgattttattttctgtttctcttctttccacctacatacatcccaaaatttagggtgcaattttaagtttattaacacatgtttttagctgcatgctgcctttgtgtgtgctcaccaaattgcattcttctctttatatgttgtatttgaattttcacaccatatgtaaacaagattacgtacgtgtccatgatcaaatacaaatgctgtcttatactggcaatttgataaacagccgtccattttttctttttctctttaactatatatgctctagaatctctgaagattcctctgccatcgaatttctttcttggtaacaacgtcgtcgttatgttattattttattctatttttattttatcatatatatttcttattttgttcgaagtatgtcatattttgatcgtgacaattagattgtcatgtaggagtaggaatatcactttaaaacattgattagtctgtaggcaatattgtcttctttttcctcctttattaatatattttgtcgaagttttaccacaaggttgattcgctttttttgtccctttctcttgttctttttacctcaggtattttagtctttcatggattataagatcactgagaagtgtatgcatgtaatactaagcaccatagctgttctgcttgaatttatttgtgtgtaaattgtaatgtttcagcgttggctttccctgtagctgctacaatggtactgtatatctattttttgcattgttttcattttttcttttacttaatcttcattgctttgaaattaataaaacaatataatatagtttgaactttgaactattgcctattcatgtaattaacttattcactgactcttattgtttttctggtagaattcattttaaattgaaggataaattaagaggcaatacttgtaaattgacctgtcataattacacaggaccctgttttgtgcctttttgtctctgtctttggttttgcatgttagcctcacacagatatttagtagttgttctgcatacaagcctcacacgtatactaaaccagtggacctcaaagtcatggccttacacctattgcatgcgagtctgtgacacaacccctggtttccatattgcaatgtgctacgccgtcgtccttgtttgtttccatatgtatattgataccatcaaattattatatcatttatatggtctggaccattacgtgtactctttatgacatgtaattgagttttttaattaaaaaaatcaatgaaatttaactacgtagcatcatatagagataattgactagaaatttgatgacttattctttcctaatcatattttcttgtattgatagccccgctgtcccttttaaactcccgagagagtataaaactgcatcgaatattacaagatgcactcttgtcaaatgaagggggggaaatgatactacaagccactaggcatggtatgatgctaaattaaattgtgcctgcaccccaggatatttcatgtgggattcatcatttattgaggaaaactctccaaattgaatcgtgcatttatattttttttccatttctagatttcttgaaggcttatggtataggcacctacaattatcagcacttctctctattgataaacaattggctgtaataccacagtagagaacgatcacaacattttgtgctggttaccttttgttttatggtcatgatttcactctctctaatctgtcacttccctccattcattttgtacttctcatatttttcacttcctggttgaaaattgtagttctcttggtacatactagtattagacattcagcaacaacaactgaactgaacttctttatactttgacacagggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcattggctttcattttctacattgccaccacctacttccacctcctccctcaccccttttccctcattgcatggccaatc

SEQ ID NO: 20 - DNA, 44 bp, Artificial sequence
  PCR primer
cuacuacuacuactcgagacaaagcctttagcctttagcctatg

SEQ ID NO: 21 - DNA, 36 bp, Artificial sequence
  PCR primer
caucaucaucauggatcccatgtctctctatgcaag

SEQ ID NO: 22 - DNA, 1704 bp, Glycine max
actatagggcacgcgtggtcgacggcccgggctggtcctcggtgtgactcagccccaagtgacgccaaccaaacgcgtcctaactaaggtgtagaagaaacagatagtatataagtataccatataagaggagagtgagtggagaagcacttctcctttttttttctctgttgaaattgaaagtgttttccgggaaataaataaaataaattaaaatcttacacactctaggtaggtacttctaatttaatccacactttgactctatatatgttttaaaaataattataatgcgtacttacttcctcattatactaaatttaacatcgatgattttattttctgtttctcttctttccacctacatacatcccaaaatttagggtgcaattttaagtttattaacacatgtttttagctgcatgctgcctttgtgtgtgctcaccaaattgcattcttctctttatatgttgtatttgaattttcacaccatatgtaaacaagattacgtacgtgtccatgatcaaatacaaatgctgtcttatactggcaatttgataaacagccgtccattttttctttttctctttaactatatatgctctagaatctctgaagattcctctgccatcgaatttctttcttggtaacaacgtcgtcgttatgttattattttattctatttttattttatcatatatatttcttattttgttcgaagtatgtcatattttgatcgtgacaattagattgtcatgtaggagtaggaatatcactttaaaacattgattagtctgtaggcaatattgtcttctttttcctcctttattaatatattttgtcgaagttttaccacaaggttgattcgctttttttgtccctttctcttgttctttttacctcaggtattttagtctttcatggattataagatcactgagaagtgtatgcatgtaatactaagcaccatagctgttctgcttgaatttatttgtgtgtaaattgtaatgtttcagcgttggctttccctgtagctgctacaatggtactgtatatctattttttgcattgttttcattttttcttttacttaatcttcattgctttgaaattaataaaacaatataatatagtttgaactttgaactattgcctattcatgtaattaacttattcactgactcttattgtttttctggtagaattcattttaaattgaaggataaattaagaggcaatacttgtaaattgacctgtcataattacacaggaccctgttttgtgcctttttgtctctgtctttggttttgcatgttagcctcacacagatatttagtagttgttctgcatacaagcctcacacgtatactaaaccagtggacctcaaagtcatggccttacacctattgcatgcgagtctgtgacacaacccctggtttccatattgcaatgtgctacgccgtcgtccttgtttgtttccatatgtatattgataccatcaaattattatatcatttatatggtctggaccattacgtgtactctttatgacatgtaattgagttttttaattaaaaaaatcaatgaaatttaactacgtagcatcatatagagataattgactagaaatttgatgacttattctttcctaatcatattttcttgtattgatagccccgctgtcccttttaaactcccgagaga

SEQ ID NO: 23 - DNA, 4010 bp, Glycine max
acaaagcctttagcctatgctgccaataatggataccaacaaaagggttcttcttttgattttgatcctagcgctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattggtaataatttttgtgtttcttactcttttttttttttttttgtttatgatatgaatctcacacattgttctgttatgtcatttcttcttcatttggctttagacaacttaaatttgagatctttattatgtttttgcttatatggtaaagtgattcttcattatttcattcttcattgattgaattgaacagtggccatggaagcttttcagatagccctttgctgaatagcctggtgggacacatcttgcattcctcaattcttgtgccataccatggatggttagttcatactggcttttttgtttgttcatttgtcattgaaaaaaaatcttttgttgattcaattatttttatagtgtgtttggaagcccgtttgagaaaataagaaatcgcatctggaatgtgaaagttataactatttagcttcatctgtcgttgcaagttcttttattggttaaatttttatagcgtgctaggaaacccattcgagaaaataagaaatcacatctggaatgtgaaagttataactgttagcttctgagtaaacgtggaaaaaccacattttggatttggaaccaaattttatttgataaatgacaaccaaattgattttgatggattttgcaggagaattagccacagaactcaccatgaaaaccatggacacattgagaaggatgagtcatgggttccagtatgtgattaattgcttctcctatagttgttcttgattcaattacattttatttatttggtaggtccaagaaaaaagggaatctttatgcttcctgaggctgttcttgaacatggctcttttttatgtgtcattatcttagttaacagagaagatttacaagaatctagacagcatgacaagactcattagattcactgtgccatttccatgtttgtgtatccaatttatttggtgagtgattttttgacttggaagacaacaacacattattattataatatggttcaaaacaatgactttttctttatgatgtgaactccattttttagttttcaagaagccccggaaaggaaggctctcacttcaatccctacagcaatctgtttccacccagtgagagaaaaggaatagcaatatcaacactgtgttgggctaccatgttttctctgcttatctatctctcattcattaactagtccacttctagtgctcaagctctatggaattccatattgggtaactaaattactcctacattgttactttttcctccttttttttattatttcaattctccaattggaaatttgaaatagttaccataattatgtaattgtttgatcatgtgcagatgtttgttatgtggctggactttgtcacatacttgcatcaccatggtcaccaccagaaactgccttggtaccgcggcaaggtaacaaaaataaatagaaaatagtgggtgaacacttaaatgcgagatagtaatacctaaaaaaagaaaaaaatataggtataataaataatataactttcaaaataaaaagaaatcatagagtctagcgtagtgtttggagtgaaatgatgttcacctaccattactcaaagattttgttgtgtcccttagttcattcttattattttacatatcttacttgaaaagactttttaattattcattgagatcttaaagtgactgttaaattaaaataaaaaacaagtttgttaaaacttcaaataaataagagtgaagggagtgtcatttgtcttctttcttttattgcgttattaatcacgtttctcttctcttttttttttttcttctctgctttccacccattatcaagttcatgtgaagcagtggcggatctatgtaaatgagtggggggcaattgcacccacaagattttattttttatttgtacaggaataataaaataaaactttgcccccataaaaaataaatattttttcttaaaataatgcaaaataaatataagaaataaaaagagaataaattattattaattttattattttgtactttttatttagtttttttagcggttagatttttttttcatgacattatgtaatcttttaaaagcatgtaatatttttattttgtgaaaataaatataaatgatcatattagtctcagaatgtataaactaataataattttatcactaaaagaaattctaatttagtccataaataagtaaaacaagtgacaattatattttatatttacttaatgtgaaataatacttgaacattataataaaacttaatgacaggagatattacatagtgccataaagatattttaaaaaataaaatcattaatacactgtactactatataatattcgatatatatttttaacatgattctcaatagaaaaattgtattgattatattttattagacatgaatttacaagccccgtttttcatttatagctcttacctgtgatctattgttttgcttcgctgtttttgttggtcaagggacttagatgtcacaatattaatactagaagtaaatatttatgaaaacatgtaccttacctcaacaaagaaagtgtggtaagtggcaacacacgtgttgcatttttggcccagcaataacacgtgtttttgtggtgtactaaaatggacaggaatggagttatttaagaggtggcctcaccactgtggatcgtgactatggttggatcaataacattcaccatgacattggcacccatgttatccaccatcttttcccccaaattcctcattatcacctcgttgaagcggtacattttattgcttattcacctaaaaacaatacaattagtacatttgttttatctcttggaagttagtcattttcagttgcatgattctaatgctctctccattcttaaatcatgttttcacacccacttcatttaaaataagaacgtgggtgttattttaatttctattcactaacatgagaaattaacttatttcaagtaataattttaaaatatttttatgctattattttattacaaataattatgtatattaagtttattgattttataataattatattaaaattatatcgatattaatttttgattcactgatagtgttttatattgttagtactgtgcatttattttaaaattggcataaataatatatgtaaccagctcactatactatactgggagcttggtggtgaaaggggttcccaaccctcctttctaggtgtacatgctttgatacttctggtaccttcttatatcaatataaattatattttgctgataaaaaaacatggttaaccattaaattctttttttaaaaaaaaaactgtatctaaactttgtattattaaaaagaagtctgagattaacaataaactaacactcatttggattcactgcagacacaagcagcaaaaccagttcttggagattactaccgtgagccagaaagatctgcgccattaccatttcatctaataaagtatttaattcagagtatgagacaagaccacttcgtaagtgacactggagatgttgtttattatcagactgattctctgctcctccactcgcaacgagactgagtttcaaactttttgggttattatttattgattctagctactcaaattactttttttttaatgttatgttttttggagtttaacgttttctgaacaacttgcaaattacttgcatagagagacatgg

SEQ ID NO: 24 - DNA, 34 bp, Artificial sequence
  PCR primer
acgaattcctcgaggtaaattaaattgtgcctgc

SEQ ID NO: 25 - DNA, 33 bp, Artificial sequence
  PCR primer
gcgagatctatcgatctgtgtcaaagtataaac

SEQ ID NO: 26 - DNA, 19 bp, Artificial sequence
  PCR primer
catgctttctgtgcttctc

SEQ ID NO: 27 - DNA, 19 bp, Artificial sequence
  PCR primer
gttgatccaaccatagtcg

SEQ ID NO: 28 - DNA, 36 bp, Artificial sequence
  PCR primer
gcgatcgatgtatgatgctaaattaaattgtgcctg

SEQ ID NO: 29 - DNA, 30 bp, Artificial sequence
  PCR primer
gcggaattcctgtgtcaaagtataaagaag

SEQ ID NO: 30 - DNA, 30 bp, Artificial sequence
  PCR primer
gatcgatgcccggggtaataatttttgtgt

SEQ ID NO: 31 - DNA, 29 bp, Artificial sequence
  PCR primer
cacgcctcgagtgttcaattcaatcaatg

SEQ ID NO: 32 - DNA, 24 bp, Artificial sequence
  PCR primer
cactcgagttagttcatactggct

SEQ ID NO: 33 - DNA, 25 bp, Artificial sequence
  PCR primer
cgcatcgattgcaaaatccatcaaa

SEQ ID NO: 34 - DNA, 38 bp, Artificial sequence
  PCR primer
cuacuacuacuactcgagcgtaaatagtgggtgaacac

SEQ ID NO: 35 - DNA, 41 bp, Artificial sequence
  PCR primer
caucaucaucauctcgaggaattcgtccattttagtacacc

SEQ ID NO: 36 - DNA, 39 bp, Artificial sequence
  PCR primer
cuacuacuacuactcgaggcgcgtacattttattgctta

SEQ ID NO: 37 - DNA, 41 bp, Artificial sequence
  PCR primer
caucaucaucauctcgaggaattctgcagtgaatccaaatg

SEQ ID NO: 38 - DNA, 22 bp, Artificial sequence
  PCR primer
caccatggtcatcatcagaaac

SEQ ID NO: 39 - DNA, 22 bp, Artificial sequence
  PCR primer
tcacgatccacagttgtgagac

SEQ ID NO: 40 - DNA, 4086 bp, Glycine max
  soybean FATB genomic clone
ttagggaaacaacaaggacgcaaaatgacacaatagcccttcttccctgtttccagcttttctccttctctctctccatcttcttcttcttcttcactcagtcaggtacgcaaacaaatctgctattcattcattcattcctctttctctctgatcgcaaactgcacctctacgctccactcttctcattttctcttcctttctcgcttctcagatccaactcctcagataacacaagaccaaacccgctttttctgcatttctagactagacgttctaccggagaaggttctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaaggtgggagggaaaatgtattttttttttctcttctaacttgcgtatattttgcatgcagcgaccttagaaattcattatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaggcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagtaagtccgtcctcatacaagtgaatctttatgatcttcagagatgagtatgctttgactaagatagggctgtttatttagacactgtaattcaatttcatatatagataatatcattctgttgttacttttcatactatatttatatcaactatttgcttaacaacaggaaactgcacttaatcatgttaaaagtgctgggcttcttggtgatggctttggttccacgccagaaatgtgcaaaaagaacttgatatgggtggttactcggatgcaggttgtggtggaacgctatcctacatggttagtcatctagattcaaccattacatgtgatttgcaatgtatccatgttaagctgctatttctctgtctattttagtaatctttatgaggaatgatcactcctaaatatattcatggtaattattgagacttaattatgagaaccaaaatgctttggaaatttgtctgggatgaaaattgattagatacacaagctttatacatgatgaactatgggaaaccttgtgcaacagagctattgatctgtacaagagatgtagtatagcattaattacatgttattagataaggtgacttatccttgtttaattattgtaaaaatagaagctgatactatgtattctttgcatttgttttcttaccagttatatataccctctgttctgtttgagtactactagatgtataaagaatgcaattattctgacttcttggtgttgggttgaagttagataagctattagtattattatggttattctaaatctaattatctgaaattgtgtgtctatatttgcttcaggggtgacatagttcaagtggacacttgggtttctggatcagggaagaatggtatgcgtcgtgattggcttttacgtgactgcaaaactggtgaaatcttgacaagagcttccaggtagaaatcattctctgtaattttccttcccctttccttctgcttcaagcaaattttaagatgtgtatcttaatgtgcacgatgctgattggacacaattttaaatctttcaaacatttacaaaagttatggaaccctttcttttctctcttgaagatgcaaatttgtcacgactgaagtttgaggaaatcatttgaattttgcaatgttaaaaaagataatgaactacatattttgcaggcaaaaacctctaattgaacaaactgaacattgtatcttagtttatttatcagactttatcatgtgtactgatgcatcaccttggagcttgtaatgaattacatattagcattttctgaactgtatgttatggttttggtgatctacagtgtttgggtcatgatgaataagctgacacggaggctgtctaaaattccagaagaagtcagacaggagataggatcttattttgtggattctgatccaattctagaagaggataacagaaaactgactaaacttgacgacaacacagcggattatattcgtaccggtttaagtgtatgtcaactagtttttttgtaattgttgtcattaatttcttttcttaaattatttcagatgttgctttctaattagtttacattatgtatcttcattcttccagtctaggtggagtgatctagatatcaatcagcatgtcaacaatgtgaagtacattgactggattctggaggtatttttctgttcttgtattctaatccactgcagtccttgttttgttgttaaccaaaggactgtcctttgattgtttgcagagtgctccacagccaatcttggagagtcatgagctttcttccgtgactttagagtataggagggagtgtggtagggacagtgtgctggattccctgactgctgtatctggggccgacatgggcaatctagctcacagtggacatgttgagtgcaagcatttgcttcgactcgaaaatggtgctgagattgtgaggggcaggactgagtggaggcccaaacctatgaacaacattggtgttgtgaaccaggttccagcagaaagcacctaagattttgaaatggttaacggttggagttgcatcagtctccttgctatgtttagacttattctggcctctggggagagttttgcttgtgtctgtccaatcaatctacatatctttatatccttctaatttgtgttactttggtgggtaagggggaaaagctgcagtaaacctcattctctctttctgctgctccatatttcatttcatctctgattgcgctactgctaggctgtcttcaatatttaattgcttgatcaaaatagctaggcatgtatattattattcttttctcttggctcaattaaagatgcaattttcattgtgaacacagcataactattattcttattatttttgtatagcctgtatgcacgaatgacttgtccatccaatacaaccgtgattgtatgctccagctcag

SEQ ID NO: 41 - DNA, 109 bp, Glycine max
  FATB intron I
gtacgcaaacaaatctgctattcattcattcattcctctttctctctgatcgcaaactgcacctctacgctccactcttctcattttctcttcctttctcgcttctcag

SEQ ID NO: 42 - DNA, 836 bp, Glycine max
  FATB intron II
gttctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaggtgggagggaaaatgtattttttttttctcttctaacttgcgtatattttgcatgcag

SEQ ID NO: 43 - DNA, 169 bp, Glycine max
  FATB intron III
gtaagtccgtcctcatacaagtgaatctttatgatcttcagagatgagtatgctttgactaagatagggctgtttatttagacactgtaattcaatttcatatatagataatatcattctgttgttacttttcatactatatttatatcaactatttgcttaacaacag

SEQ ID NO: 44 - protein, 328 aa, Glycine max
  soybean FATB enzyme
MEEQLLAAITTIFLAAEKQWMMLDWKPRRPDMLIDPFGIGKIVQDGLVFRENFSIRSYEIGADRTASIETVMNHLQETALNHVKSAGLLGDGFGSTPEMCKKNLIWVVTRMQVVVERYPTWGDIVQVDTWVSGSGKNGMRRDWLLRDSKTGEILTRASSVWVMMNKLTRRLSKIPEEVRQEIGSYFVDSDPILEEDNRKLTKLDDNTADYIRTGLSPRWSDLDINQHVNNVKYIGWILESAPQPILESHELSSMTLEYRRECGRDSVLDSLTAVSGADMGNLAHSGHVECKHLLRLENGAEIVRGRTEWRPKPVNNFGVVNQVPAEST

SEQ ID NO: 45 - DNA, 1856 bp, Glycine max
  soybean FATB partial genomic clone
ttagggaaacaacaaggacgcaaaatgacacaatagcccttcttccctgtttccagcttttctccttctctctctccatcttcttcttcttcttcactcagtcaggtacgcaaacaaatctgctattcattcattcattcctctttctctctgatcgcaaactgcacctctacgctccactcttctcattttctcttcctttctcgcttctcagatccaactcctcagataacacaagaccaaacccgctttttctgcatttctagactagacgttctaccggagaaggttctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaggtgggagggaaaatgtattttttttttctcttctaacttgcgtatattttgcatgcagcgaccttagaaattcattatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaggcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagtaagtccgtcctcatacaagtgaatctttatgatcttcagagatgagtatgctttgactaagatagggctgtttatttagacactgtaattcaatttcatatatagataatatcattctgttgttacttttcatactatatttatatcaactatttgcttaacaacaggaaactgcacttaatcatgttaaaagtgctgggcttcttggtgatggctggta

SEQ ID NO: 46 - DNA, 34 bp, Artificial
  Oligonucleotide primer F1
gcggccgccccgggttagggaaacaacaaggacg

SEQ ID NO: 47 - DNA, 34 bp, Artificial
  Oligonucleotide primer F2
gcggccgccccgggcagtcagatccaactcctca

SEQ ID NO: 48 - DNA, 34 bp, Artificial
  Oligonucleotide primer F3
gcggccgccccgggattggtgctgatcgtaccgc

SEQ ID NO: 49 - DNA, 38 bp, Artificial
  Oligonucleotide primer R1
gcggccgcggtacccccccttacccaccaaagtatcac

SEQ ID NO: 50 - DNA, 34 bp, Artificial
  Oligonucleotide primer R2
gcggccgcggtaccaaactctccccagggaacca

SEQ ID NO: 51 - DNA, 34 bp, Artificial
  Oligonucleotide primer R3
gcggccgcggtaccagccatcaccaagaagccca

SEQ ID NO: 52 - DNA, 37 bp, Artificial
  Oligonucleotide primer 18133
gaattcctcgagctcgattcttttctcttttaacttt

SEQ ID NO: 53 - DNA, 37 bp, Artificial
  Oligonucleotide primer 18134
gaattcctcgagcatgcaaaatatacgcaagttagaa

SEQ ID NO: 54 - DNA, 854 bp, Artificial
  PCR product containing soybean FATB intron II
gaattcctcgagctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaggtgggagggaaaatgtattttttttttctcttctaacttgcgtatattttgcatgctcgaggaattc

SEQ ID NO: 55 - DNA, 1688 bp, Glycine max
  soybean FATB cDNA
acaattacactgtctctctcttttccaaaattagggaaacaacaaggacgcaaaatgacacaatagcccttcttccctgtttccagcttttctccttctctctctctccatcttcttcttcttcttcactcagtcagatccaactcctcagataacacaagaccaaacccgctttttctgcatttctagactagacgttctaccggagaagcgaccttagaaattcattatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaagcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagaaactgcacttaatcatgttaaaagtgctgggcttcttggtgatggctttggttccacgccagaaatgtgcaaaaagaacttgatatgggtggttactcggatgcaggttgtggtggaacgctatcctacatggggtgacatagttcaagtggacacttgggtttctggatcagggaagaatggtatgcgtcgtgattggcttttacgtgactccaaaactggtgaaatcttgacaagagcttccagtgtttgggtcatgatgaataagctaacacggaggctgtctaaaattccagaagaagtcagacaggagataggatcttattttgtggattctgatccaattctggaagaggataacagaaaactgactaaacttgacgacaacacagcggattatattcgtaccggtttaagtcctaggtggagtgatctagatatcaatcagcatgtcaacaatgtgaagtacattggctggattctggagagtgctccacagccaatcttggagagtcatgagctttcttccatgactttagagtataggagagagtgtggtagggacagtgtgctggattccctgactgctgtatctggggccgacatgggcaatctagctcacagcgggcatgttgagtgcaagcatttgcttcgactggaaaatggtgctgagattgtgaggggcaggactgagtggaggcccaaacctgtgaacaactttggtgttgtgaaccaggttccagcagaaagcacctaagatttgaaatggttaacgattggagttgcatcagtctccttgctatgtttagacttattctggttccctggggagagttttgcttgtgtctatccaatcaatctacatgtctttaaatatatacaccttctaatttgtgatactttggtgggtaagggggaaaagcagcagtaaatctcattctcattgtaattaaaaaaaaaaaaaa